(19) United States
(12) Patent Application Publication
(10) Pub. No.: US 2009/0010941 A1
(43) Pub. Date: Jan. 8, 2009

(54) Methods for treating HIV

(75) Inventors: Mario Stevenson, Worcester, MA (US); Simon Swingler, Worcester, MA (US)

Correspondence Address: WOLF GREENFIELD & SACKS, P.C., 600 ATLANTIC AVENUE, BOSTON, MA, 02210-2206, US

(73) Assignee: University of Massachusetts, Boston, MA (US)

(21) Appl. No.: 12/082,092

(22) Filed: Apr. 8, 2008

Related U.S. Application Data
(60) Provisional application No. 60/922,483, filed on Apr. 9, 2007.

(51) Int. Cl. A61K 39/395; C12N 5/06; A61K 31/7105; A61K 31/19; A61K 31/185; A61K 31/7048; A61P 31/18; A61K 38/02

(52) U.S. Cl. 424/158.1; 435/375; 514/44; 514/557; 514/575; 514/27; 514/2

(57) ABSTRACT

The invention relates to methods of treating HIV by administering a TRAIL receptor activator. The invention also relates to methods for inducing apoptosis in an HIV reservoir cell by contacting the cell with TRAIL receptor activator such as an M-CSF effector kinase inhibitor.

### RELATED APPLICATION

This application claims priority under 35 U.S.C. § 119 to U.S. Provisional Application No. 60/922,483, filed Apr. 9, 2007, the entire contents of which is hereby incorporated by reference.

### GOVERNMENT SUPPORT

The work resulting in this invention was supported in part by NIH grants RR11589 and MH64411. The U.S. Government may therefore be entitled to certain rights in the invention.

### BACKGROUND OF THE INVENTION

Infectious viruses prolong the survival of infected cells to allow full maturation and dissemination. On the other hand, apoptosis of the infected cells triggered by either immune attack or as a direct outcome of viral infection is an important component of the host antiviral response. Therefore, it is not surprising that many viruses encode viral proteins that inhibit this process. There are two main pathways for induction of apoptotic cell death. One involves death receptors on the cell surface (TNF receptor family), TNF-R1, Fas, DR4, and DR5 by interaction with their respective ligands TNF-α, Fas ligand (FasL), and TRAIL. The second pathway involves the participation of mitochondria and is regulated by members of Bcl-2 family including the antiapoptotic molecules, bcl-2 or bcl-XL, or by the proapoptotic effectors, bad, bax, or bak. Both death receptor and mitochondria pathways share a common set of caspases that cleave specific cellular substrates, leading to DNA fragmentation, the hallmark of apoptosis. Viruses can block one or both pathways (Xiao-Ning Xu, Gavin R. Screaton, and Andrew J. McMichael; 2001. Virus Infections: Escape, Resistance, and Counterattack. Immunity, 15: 867-870).

### SUMMARY OF THE INVENTION

In one aspect, the invention provides methods for inducing apoptosis in an HIV reservoir cell, such as a macrophage. This is at least in part mediated by altering the resistance of HIV-1 envelope-expressing reservoir cells to apoptosis. The methods involve contacting the HIV reservoir cell with an effective amount of an M-CSF effector kinase inhibitor for inhibiting M-CSF signaling. In some cases, the methods may further include contacting the HIV reservoir cell with a TRAIL molecule, e.g., a TRAIL agonist.

In some embodiments, the effective amount of the M-CSF effector kinase inhibitor is not an effective amount to inhibit HIV replication. In some embodiments, the M-CSF effector kinase inhibitor may be selected from: Gleevec® (Imatinib mesylate; ST1571); Nilotinib; Dasatinib; Sorafenib; Sunitinib; GW2580, 5-{3-methoxy-4-[(4methoxybenzyl)oxy]benzyl}pyrimidine-2,4-diamine; ABT-869; AG013736; BAY 43-9006; CHIR258; and SU11248.

In some embodiments, the methods for inhibiting the M-CSF signaling further involve contacting the reservoir cell with an effective amount of a histone deacetylase inhibitor, such as valproic acid (VPA) (Depakote®), sulforaphane, suberoylanilide hydroxamic acid (SAHA), sodium n-butyrate, suberoylanilide hydroxamic acid, LAQ824, CI-994, MS-275, and depsipeptide.

According to another aspect of the invention, methods for treating a subject having an HIV infection are provided. These methods involve administering to a subject in need of such treatment an effective amount of an M-CSF effector kinase inhibitor for inhibiting M-CSF signaling, wherein the effective amount of M-CSF effector kinase inhibitor is not an effective amount for inhibiting HIV replication.

In some embodiments, the effective amount of M-CSF effector kinase inhibitor is not an effective amount for inhibiting NFκB activity or expression.

In some embodiments, the effective amount of an M-CSF effector kinase inhibitor induces TRAIL-mediated apoptosis in an HIV reservoir cell and causes a reduction in a number of HIV reservoir cells in the subject.

Yet in another aspect, the invention includes methods for treating a subject having an HIV infection. The methods involve administering to a subject in need of such treatment an effective amount for treating HIV of a histone deacetylase inhibitor and a TRAIL receptor activator. TRAIL receptor activators include, but are not limited to: an M-CSF antagonist (e.g., an antibody to M-CSF, an siRNA for M-CSF and an M-CSF effector kinase inhibitor); a TRAIL receptor agonist; and a TRAIL receptor expression construct.

In some embodiments of the methods provided herein, the histone deacetylase inhibitor may be: valproic acid (VPA) (Depakote®), sulforaphane, suberoylanilide hydroxamic acid (SAHA), sodium n-butyrate, suberoylanilide hydroxamic acid, LAQ824, CI-994, MS-275, and/or depsipeptide.

In a further aspect, the invention includes methods for upregulating cell surface death receptors, such as DR4 and/or DR5, in an HIV-infected reservoir cell. The methods involve contacting the HIV-infected reservoir cell with an effective amount of an M-CSF antagonist to upregulate cell surface death receptors, and contacting the cell with TRAIL ligand or an agonist thereof (e.g., a TRAIL receptor antibody). The M-CSF antagonist of the methods may be selected from, for example: an M-CSF antibody, siRNA for M-CSF, Gleevec® (Imatinib mesylate; ST1571); Nilotinib; Dasatinib; Sorafenib; Sunitinib; GW2580, 5-{3-methoxy-4[(4methoxybenzyl)oxy]benzyl}pyrimidine-2,4-diamine; ABT-869; AG013736; BAY 43-9006; CHIR258; and SU11248.

In still further aspect, the invention provides methods for treating a subject having an HIV infection by selectively inducing TRAIL-mediated apoptosis in an HIV-infected cell, the method comprising administering to a subject in need of such treatment a TRAIL receptor activator, which may include a TRAIL receptor gene expression construct and/or a topoisomerase II inhibitor (e.g., Etoposide (Eposin®, Etopophos®, Vepesid®, VP-16®) or teniposide).

In yet another aspect, the invention includes methods for treating an HIV-infected subject using one or more agents that regulate one or more molecular targets that regulate apoptosis. The methods contemplate using one or more agents that regulate apoptotic targets, including but not limited to: Death receptors, CD95/Fas, TNF, Caspases, IAPs/SMAC, Bcl-2 and p53. In some embodiments, one or more of the agents that regulate these targets are used in combination with an M-CSF inhibitor to treat an HIV-infected subject.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### BRIEF DESCRIPTIONS OF THE DRAWINGS

FIG. 1 provides two sets of graphs showing HIV-1 envelope-dependent M-CSF induction by infected macrophages. Results were obtained with macrophages from three independent donors. The first set (upper panels) shows levels of virus as measured by reverse transcriptase activity. The second set (lower panels) shows levels of M-CSF production by macrophages. Data were obtained following infection with VSV-envelope-pseudotyped X4 HIV-1 variants (HIV-1 LAI) containing intact (WT) or defective (Δenv) envelope genes.

FIG. 2 provides a set of data showing that MCSF inhibition does not influence virus production by infected macrophages. FIG. 2A depicts virus production in the presence of blocking antibodies to M-CSF. Macrophages were infected with pseudotyped HIV-1 LAI and maintained in the presence of M-CSF-neutralizing or isotype (Iso) antibodies and at the indicated intervals post-infection, culture supernatants were examined for virus production (left panel) and M-CSF (right panel). FIG. 2B is a schematic showing the time course of experiments and data collection. FIG. 2C shows virus production after inhibition of M-CSF production by RNAL Macrophages were infected with an R5 HIV-1 variant (HIV-1_{ADA}) and monitored for virus and M-CSF production. Macrophages were transfected with M-CSF-specific or scrambled (Scr) siRNAs at days 6 and 7 post infection. 24 hours later, the extent of MCSF silencing was determined by analysis of M-CSF transcripts (FIG. 2D) and M-CSF production (FIG. 2E) and virus output determined from RT activity in culture supernatants (FIG. 2F).

FIG. 3 provides three sets of data demonstrating that M-CSF and HIV-1 envelope regulate TRAIL-R1 expression on infected macrophages. FIG. 3A is a graph showing death receptor gene expression in macrophages infected with pseudotyped wildtype or envelope minus HIV-1 variants. Messenger RNA levels were determined by densitometry of cDNA gene arrays and data plotted relative to HIV-1 LAI wild type infected cells (Error bars, SD). FIG. 3B provides flow cytometry data showing cell surface expression of death receptors. HIV-1 infected cells were identified in flow cytometry by expression of muCD24 (HSA) cloned in place of viral Vpr gene and the levels of TRAIL-R1, Fas and TWEAK-R determined. FIG. 3C is a graph demonstrating modulation of TRAIL-R1 expression by M-CSF. Data from flow cytometry on macrophages infected with pseudotyped HIV-1 HSA WT or Δenv viruses after treatment with M-CSF or neutralizing antibody to M-CSF for 16 hours. (Error bars, SD).

FIG. 4. provides three sets of data to demonstrate resistance of macrophages to TRAIL-mediated apoptosis and enhanced longevity in the presence of TRAIL depend upon an intact HIV-1 envelope gene. Macrophages were infected with pseudotyped wild-type or envelope-minus HIV-1 LAI variants and monitored for virus production (FIG. 4A). At 8 days post infection, cultures were incubated with soluble TRAIL (100 ng·ml-1) and apoptosis determined by ELISA for active (cleaved) Caspase 3. Images of macrophages shown in FIG. 4B show that TRAIL-induced morphological changes in macrophages infected with Δenvelope HIV-1. Macrophage cultures, equally infected with pseudotyped HIV-1 LAI WT or Δenv viruses or mock infected, were examined by phase contrast microscopy after incubation with 100 ng/ml TRAIL (1 hour treatment with TRAIL followed by 5 hours incubation after removal of TRAIL). FIG. 4C shows a graph showing increased longevity of infected macrophages by envelope in the presence of TRAIL was observed. Eight days after infection with pseudotyped HIV-1 LAI WT or Δenv viruses, macrophages were incubated with soluble TRAIL (100 ng·ml-1) and percentage of viable cells remaining extrapolated from the level of cell death determined from ELISA. Macrophage half-life was calculated from the linear regression slope and expressed in hours+/−SD.

FIG. 5. provides three graphs showing that cell viability and virus production in the presence of TRAIL requires an intact envelope gene and is augmented by M-CSF. Macrophage viability and virus production in the presence of increasing concentrations of TRAIL is shown in FIG. 5A and FIG. 5B, respectively. Four days after infection with pseudotyped HIV-1 LAI WT or Δenv viruses, macrophages were incubated with increasing concentrations of soluble TRAIL and cell viability (FIG. 5A) and virus production (FIG. 5B) determined after 16 hours by MTT assay and reverse transcriptase production, respectively. Activated CD8+ T cells suppress viral replication in macrophages via TRAIL (FIG. 5C). Macrophages were infected with pseudotyped wild-type or envelope-minus HIV-1 LAI variants and monitored for virus production in a 24 hour period after incubation with Anti-CD3/CD28 stimulated autologous CD8+ TRAIL+T lymphocytes for 4 hours. Macrophages were pre-treated for 16 hours with M-CSF neutralizing antibody or isotype control and T cells were preincubated with recombinant TRAIL-R1 or control receptor (5 μg·ml-1) for 1 hour.

FIG. 6. provides three graphs that demonstrate that Wild type infected macrophages retain substantial resistance to TRAIL even when M-CSF output is low. Relationship of apoptosis by TRAIL, M-CSF release and TRAIL-R1 expression in macrophages infected with pseudotyped wild type or envelope minus HIV-1 HSA viruses. In FIG. 6A, M-CSF release as a function of time was determined by ELISA. In FIG. 6B, TRAIL-R1 expression as a function of time was measured by flow cytometry. In FIG. 6C, TRAIL-mediated apoptosis in macrophages was analyzed by ELISA for active (cleaved) Caspase 3 in parallel cultures during the infection (Error bars, SD).

FIG. 7 provides graphs showing envelope up-regulated M-CSF and anti-apoptotic genes combine to negate TRAIL-dependent apoptosis in HIV-1 infected macrophages. In FIG. 7A, messenger RNA (mRNA) levels for the anti-apoptotic genes, cIAP-1, cIAP-2, XIAP, Bfl-1 and Mcl-1 were measured by real-time PCR during infection of macrophage cultures infected with pseudotyped HIV-1 LAI WT and Δenv viruses. M-CSF release was determined by ELISA. At seven days post infection when viral replication and MCSF production approached peak levels, macrophages cultures were transfected with Mcl-1, Bfl-1, cIAP-1, -2 and XIAP siRNAs or a non-targeting (SCR1) control. Sixteen hours later macrophages were treated with soluble TRAIL, recombinant M-CSF or a combination of both and apoptosis determined by ELISA for active (cleaved) Caspase 3 (Error bars, SD) (FIG. 7B). Mean inhibition of mRNAs was 72.0%+/−3.4% for Mcl-1, 75.0%+/−3.9% for Bfl-1 and 76.0+/−3.6% for cIAP-1, -2 and XIAP relative to SCR1 (mean+/−SD) by quantitative RT-PCR.

FIG. 8 provides two graphs showing that envelope protects macrophages from TRAIL-mediated apoptosis and permits highly efficient infection of CD4+ T lymphocytes in the presence of TRAIL+cytotoxic T cells. As shown in FIG. 8A, macrophages were infected with pseudotyped HIV-1 LAI WT and Δenv viruses and at 5 days post infection, when viral replication and M-CSF production approached peak levels, M-CSF was neutralized by specific antibody and the anti-apoptotic genes, cIAP-1, cIAP2, XIAP, Bfl-1 and Mcl-1 were silenced by RNA interference. Specific siRNA treatment is indicated by ALL and non-specific siRNA treatment by SCR1. Autologous T lymphocytes, previously activated by anti-CD3/CD28 antibodies, were incubated with recombinant TRAIL-R1 or control receptor for 1 hour. Efficiency of viral dissemination in trans to T lymphocytes after 4 hours co-culture was determined by quantitation of episomal viral cDNA transcripts in isolated lymphocytes after normalization to CCR5 levels. Infection in trans is shown as the percentage of viral cDNAs in T lymphocytes relative to co-cultures were M-CSF and anti-apoptotic genes remained unmodulated. (Error bars, indicate SD). Mean siRNA mediated inhibition of all antiapoptotic genes (ALL) was 68.8%+/−2.0% relative to SCR1 (mean+/−SD) by quantitative RT-PCR. In FIG. 8B, macrophage cultures were infected and treated with neutralizing antibody to M-CSF or RNA interference to anti-apoptotic genes as described in FIG. 8A, except HIV-1 variants encoding HSA were employed for infection. Following 4 hour co-culture with anti-CD3/CD28 stimulated autologous TRAIL+T lymphocytes, macrophage caspase activation was measured by flow cytometry in CD14+HIV-1 infected cells using the fluorescent probe Red-VAD-FMK. Caspase activation is shown as fold increase relative to HIV-1 wild type infected macrophages were M-CSF and anti-apoptotic genes were unmodulated (Error bars, SD).

FIG. 9 provides two images of protein immunoblots and two sets of flow cytometry data, showing that the anti-leukemia drug, Imatinib, blocks M-CSF signaling and up-regulates TRAIL-R1 expression on infected macrophages to render those cells susceptible to apoptosis by TRAIL. For FIG. 9A, macrophages were incubated for 16 hours with 2 μM Imatinib mesylate before stimulation with recombinant M-CSF for 5 minutes. The M-CSF receptor was immunoprecipited from cell lysates and M-CSF dependent tyrosine autophosphorylation determined by Western blotting and densitometry. For FIG. 9B, macrophages were infected with pseudotyped HIV-1 HSA WT and Δenv viruses and at 8 days post infection, when viral replication and M-CSF production approached peak levels, macrophages were incubated with Imatinib for 16 hours and TRAIL-R1 levels determined by flow cytometry. For FIG. 9C, macrophage cultures replicating pseudotyped HIV-1 LAI WT virus, as in FIG. 9B, were incubated with Imatinib for 16 hours then challenged with soluble TRAIL. Apoptosis in HIV-1 infected and uninfected cells, from the same culture, were measured 6 hours later by Annexin V and propidium iodide staining and flow cytometry. Apoptotic cells were Annexin V+, propidium iodide-(lower right quadrant).

FIG. 10 provides four graphs that depict measurements of reverse transcriptase activities and M-CSF. M-CSF induction by a CD4-binding mutant of HIV-1 was examined by assaying for Virus production (FIG. 10A) and M-CSF release (FIG. 10B). Data were collected at various intervals following infection with pseudotyped HIV-1 variants containing intact or deleted envelope genes or an HIV-1 variant (HIV-1 LAI ΔCD4b) lacking a functional CD4 receptor binding motif in envelope. Cumulative M-CSF release (shown in FIG. 10C) during the course of viral replication was determined by normalizing the amount of M-CSF to Reverse Transcriptase output and illustrated by bar chart (error bars, SD). FIG. 10D shows effect of cell free HIV-1 virions on M-CSF production by macrophages. R5-tropic HIV-1ADAWT and VSV-pseudotyped HIV-1 ADA Δenv virions were purified on a continuous 15-60% sucrose gradients. Individual gradient fractions were dialyzed, analyzed for reverse transcriptase activity and added to macrophage cultures for 1 hour and M-CSF production determined after 16 hours later. Gradient fractions of mock-infected macrophage supernatants were used as controls.

FIG. 11 provides a set of six protein immunoblot images. The following four conditions were tested: macrophages infected with pseudotyped HIV-1 LAI WT, with (second lane from left) or without (left lane) Imatinib treatment; macrophages infected with pseudotyped HIV-1 LAI Δenvelope (third lane); and mock treated macrophages (right lane). Protein levels of Bcl-2, Mcl-1, BClx_{L}, BimL, Bmf and cFLIP were determined by Western blotting. Imatinib was used at a concentration of 2 μM for 16 hours.

FIG. 12 provides a graph showing DNA fragmentation of HIV-infected macrophages measured at 0, 1 and 5 days after Imatinib treatment.

FIG. 13 provides a graph showing relative mRNA expression of 12 genes that are involved in apoptotic pathways. The data show that HIV-1 envelope regulates host genes involved in apoptosis.

FIG. 14 illustrates a schematic of Experimental Model. The envelope protein, produced de novo in macrophages, provides resistance to TRAIL-mediated apoptosis through multiple mechanisms: the induction of M-CSF and the subsequent down-regulation of Death Receptor for TRAIL-R1, and the M-CSF and envelope dependent up-regulation of several anti-apoptotic genes. These defensive processes permit HIV-1 infected macrophages to survive the hostile microenvironment created by TRAIL bearing cytotoxic T lymphocytes and maintain high level virus production. Envelope substantially reduces TRAIL receptor expression and provides inhibition of both the extrinsic caspase activation and intrinsic mitochondrial damage pathways following signals emanating from TRAIL receptors. Such a mechanism allows infected macrophages to withstand the cell contacts that occur during cell-to-cell contact and efficiently transmit virus to CD4+ lymphocytes.

### DETAILED DESCRIPTION OF THE INVENTION

Evidence suggests that macrophages may serve as an HIV reservoir in an infected subject and present great risk to the pathogenesis of HIV infection (thus AIDS). The turnover of infected macrophages, which has been extrapolated from decay characteristics of plasma virions during highly active antiretroviral therapy (HAART) and from reported estimates of macrophage life span in the tissues (Perelson et al., 1996), is slow (half-life of 2-4 weeks). Moreover, a suggested half-life of 2-4 weeks may be an underestimation; indeed, significantly greater half-life is suggested by studies with highly pathogenic SHIV variants which demonstrate that tissue macrophages can sustain viremia for prolonged intervals (Igarashi et al., 2001).

The instant invention provides a mechanism whereby cytokine induction by the HIV-1 envelope glycoprotein protects infected macrophages from death receptor-mediated killing. Evidence presented herein demonstrates that the envelope glycoprotein of HIV-1 induces production of the cytokine, monocyte colony stimulating factor (MCSF), which in turn restricts the expression of the death receptors TRAIL DR4 and TRAIL DR5 on infected macrophages, rendering them resistant to TRAIL-induced apoptosis. In contrast, macrophages infected with an envelope-minus HIV-1 variant were highly sensitive to killing by TRAIL. TRAIL receptor expression on infected macrophages was increased when MCSF was inhibited by blocking antibodies or by RNA interference and exogenous MCSF conferred TRAIL resistance on macrophages infected with an envelope-minus HIV-1 variant. In the presence of an intact viral envelope, infected cell viability and virus production were sustained in the presence of high concentrations of TRAIL. Furthermore, it was demonstrated herein that the suppression of death receptors by the viral envelope counteracts TRAIL on the surface of lymphocytes, thereby promoting viral dissemination in trans in macrophage lymphocyte co-cultures. The data presented herein suggest that the HIV-1 envelope glycoprotein, which paradoxically is a major determinant of viral cytopathicity in CD4+ lymphocytes, ensures infected macrophage survival and viral dissemination in the face of host apoptotic clearance processes.

The invention, therefore, is based at least in part on the surprising finding that HIV-1 envelope can control cellular resistance of infected reservoir cells to apoptosis in an M-CSF-dependent manner. Thus, the methods provided in the invention are useful for inducing apoptosis specifically in infected cells that express the M-CSF receptor, c-fms. The invention in some aspects involves treatment of an HIV-infected subject with agents that activate apoptotic pathways in HIV-infected reservoir cells. The compounds used in the invention provide a novel strategy for effective reservoir depletion.

The present invention is based in part on a recognition that a chronic infection, particularly that of a retrovirus such as HIV, relates at least in part to the virus' ability to persist in reservoir cells, such as macrophages, which then are able to cause further dissemination in a host organism. Furthermore, the invention is based in part on the finding that HIV-1 envelope protein has novel functions that protect macrophages from TRAIL-mediated apoptosis by inducing anti-apoptosis genes and the cytokine M-CSF, which down-regulates TRAIL receptors. These functions are suited for HIV dissemination from macrophage reservoirs to CD4+ and/or CD8+ T lymphocytes. The survival of infected macrophages during interactions with TRAIL-expressing cells likely increases macrophage persistence and CD4+ T cell depletion through enhanced viral transmission during HIV-1 disease. CD8 T cells, Natural Killer cells and CD4 T cells all may contribute to TRAIL-dependent apoptosis of macrophages in vivo. Specifically targeting the M-CSF receptor will negate some or most of the resistance to TRAIL-mediated apoptosis by HIV infected macrophages.

More specifically, data presented herein demonstrate that HIV-1 envelope in infected macrophages negated apoptotic signals engendered by TRAIL. Macrophages infected with HIV-1 lacking a functional envelope were highly susceptible to TRAIL yet cell viability and virus output were sustained in wild type infected macrophages despite the presence of TRAIL. Envelope conferred TRAIL resistance via M-CSF-dependent down-regulation of TRAIL Receptor-1 and up-regulation of anti-apoptotic IAP and Bcl family genes. Inhibition of envelope's protective functions rendered wild type-infected macrophages sensitive to TRAIL and substantially reduced viral dissemination and transmission to activated T lymphocytes. As described herein, TRAIL receptor activators greatly elevated TRAIL-R1 expression in wild type infected macrophages and rendered them highly susceptible to apoptosis by TRAIL. In vivo, TRAIL receptor activators may enable immune effector cells to eliminate macrophage reservoirs of HIV-1 and prevent viral dissemination to CD4+ T cells.

Thus, in some aspects, the invention relates to methods of treating HIV by administering to a subject a TRAIL receptor activator. A TRAIL receptor activator, as used herein, is a compound that directly or indirectly activates TRAIL receptor by increasing it's expression, stability or activity. TRAIL receptor activators include but are not limited to: M-CSF antagonists, TRAIL agonists, TRAIL receptor expression constructs and a topoisomerase II inhibitor.

In certain embodiments of such methods, an effective amount of an M-CSF antagonist may be used, which acts as a TRAIL receptor activator. This is based, at least in part, on the recognition that in HIV reservoir cells, HIV-1 envelope can cause M-CSF-mediated downregulation of TRAIL receptor expression, specifically, DR4/DR5 containing death domains, rendering the infected cells unresponsive to apoptotic signals. Therefore, by disengaging the protective pathway by antagonizing the effect of M-CSF, the reservoir cells may be made more susceptible to death signals.

An “M-CSF antagonist” is a compound that interferes with M-CSF signaling activity. The M-CSF antagonist may be an expression inhibitor, such as an siRNA or antisense molecule. Alternatively, the M-CSF antagonist may alter the activity of M-CSF by interacting directly on the M-CSF molecule or interfering with M-CSF signaling.

Thus, M-CSF antagonists also include antibodies and M-CSF effector kinase inhibitors.

As used herein, the term “signaling pathway” refers to a cascade of transduction events involving an initial triggering event to the manifestation of downstream cellular effects. Thus, “M-CSF signaling pathway” shall encompass action of an M-CSF ligand and cofactors, M-CSF receptors and their downstream effectors that convey cellular effects, e.g., any factor or factors, binding partners or receptors, or effectors. Therefore, inhibition of M-CSF signaling pathway may be effectuated at multiple levels. For example, M-CSF signaling may be inhibited by interfering with the expression or activity of the ligand itself, at the receptor level, and/or at the effector level.

Macrophage Colony Stimulating Factor Receptor is the homodimeric product of the c-fms proto oncogene and maps to chromosome 5 at band 5q33.3. It is a member of the type III subfamily of receptor tyrosine kinases. Other members of this subfamily include Flt-3, the receptor for SCF, and the alpha and beta receptors for PDGF. These receptors are characterized by the presence of five immunoglobulin-like domains in their extracellular region and a split kinase domain in their intracellular region. M-CSF binding induces receptor homodimerization, resulting in transphosphorylation of specific cytoplasmic tyrosine residues and signal transduction.

Thus, in some embodiments the methods of the invention involve the use of an M-CSF antagonist that alters the activity of M-CSF by interacting indirectly with M-CSF. Antagonizing M-CSF effects may thus be achieved by interfering with its downstream signaling pathway. Accordingly, the invention also contemplates using an inhibitor that can inhibit the activity of M-CSF effector kinase or kinases. Because M-CSF receptor is a tyrosine kinase, its activation may be inhibited by a kinase inhibitor that inhibits the kinase activity associated with the receptor activation. In addition, any one of the downstream effector kinases along the signal transduction cascade may also be inhibited by such a kinase inhibitor. As described in more detail below, one consequence of the inhibition of M-CSF signaling by any one of the means described herein is the resultant upregulation of TRAIL receptors in the infected cell (e.g., a reservoir). As a result, the infected reservoir cell is more receptive to TRAIL-mediated death signals. The invention thus embraces methods for rendering an HIV reservoir cell more responsive to TRAIL-dependent death signals by antagonizing M-CSF-mediated downregulation of TRAIL receptor expression.

As used herein, “downregulation” or “down-regulation” shall mean that the expression of a molecule is reduced in a cell by one or more of the following mechanisms: downregulation may occur at the transcriptional level such that fewer mRNA transcripts are available. This may be a result of reduced transcriptional activity itself, or may be due to reduced RNA processing, such that the production of mature mRNA is suppressed or retarded. Downregulation may occur at the level of translation such that less protein is being produced from a corresponding transcript. Alternatively, while not mutually exclusive, downregulation at the protein level may also occur when the maturation of a functional protein is inhibited in some way, such as pathways involving post-translational modifications of protein that contribute to the functionality of the protein. For example, some proteins require modifications such as addition of lipid moiety that allows the protein to localize to a correct compartment of the cell. Similarly, some proteins are required to be complexed with other subunits to form a fully functional entity, e.g., ion channels. In some of these cases, subunits may become covalently coupled via disulfide bridge. Therefore, a defect or suppression of enzymes and other cellular processes that confer each of these modifications may halt the cellular availability of the target protein. In some cases, downregulation occurs when a protein is prevented from localizing to a correct subcellular compartment. This may occur in a regulated fashion, such as receptor internalization. Protein downregulation may also result from protein degradation and/or reduced protein stability.

Conversely, the term “upregulation” or “up-regulation” shall refer to the opposite effects, which result in increased expression, activity and/or availability of a molecule.

In some embodiments of the invention, the methods include one or more of M-CSF effector kinase inhibitors. An M-CSF effector kinase inhibitor is a compound that blocks or reduces the function of M-CSF effector kinase. Non-limiting examples of M-CSF effector kinase inhibitors include: Gleevec® (Imatinib mesylate; ST1571); Nilotinib; Dasatinib; Sorafenib; Sunitinib; GW2580, 5-{3-methoxy-4-[(4methoxybenzyl)oxy]benzyl}pyrimidine-2,4-diamine; ABT-869; AG013736; BAY 43-9006; CHIR258; SU11248. In some cases, the M-CSF effector kinase that is inhibited by one or more of such inhibitors is an intrinsic M-CSF receptor kinase. In some cases, the M-CSF kinase that is inhibited by one or more of such inhibitors is a kinase associated with a downstream effector of the M-CSF receptor. In some circumstances, a kinase inhibitor may effectively inhibit kinase activity associated with more than one targets of M-CSF signaling.

Imatinib is a drug used to treat certain types of cancer. It is currently marketed by Novartis as Gleevec® (USA) or Glivec® (Europe/Australia) as its mesylate salt, Imatinib mesilate (INN). It is also referred to as CGP57148B or ST1571. It is used in treating chronic myelogenous leukemia (CML), gastrointestinal stromal tumors (GISTs) and a number of other malignancies. It is the first member of a new class of agents that act by inhibiting particular tyrosine kinase enzymes, instead of non-specifically inhibiting rapidly dividing cells. Imatinib is a 2-phenylaminopyrimidine derivative that functions as a specific inhibitor of a number of tyrosine kinase enzymes. It occupies the TK active site, leading to a decrease in activity. Imatinib is specific for the TK domain in abl (the Abelson proto-oncogene), c-kit and PDGF-R (platelet-derived growth factor receptor) and other related family of proteins, including M-CSF receptor.

The methods of the invention may also involve the use of an M-CSF antagonist that alters the activity of M-CSF by interacting directly on the M-CSF molecule or interfering with M-CSF signaling. An M-CSF antagonist that interacts directly with the M-CSF and inhibits its activity works by binding to the M-CSF or its ligands and reducing signaling cascades. These M-CSF activity inhibitors include M-CSF binding molecules such as antibodies, fragments thereof, peptides, scFv, and small molecules.

In certain embodiments, the M-CSF antagonist may be an antibody or fragment thereof or single-chain variable fragment (ScFv) against M-CSF, which can neutralize the effect of M-CSF. Antibodies are well known to those of ordinary skill in the science of immunology. As used herein, the term “antibody” means not only intact antibody molecules but also fragments of antibody molecules retaining specific binding ability. Such fragments are also well known in the art and are regularly employed both in vitro and in vivo. In particular, as used herein, the term “antibody” means not only intact immunoglobulin molecules but also the well-known active fragments F(ab′)₂, and Fab. F(ab′)₂, and Fab fragments which lack the Fc fragment of intact antibody, clear more rapidly from the circulation, and may have less non-specific tissue binding of an intact antibody (Wahl et al., J. Nucl. Med. 24:316-325 (1983)).

As is well-known in the art, the complementarity determining regions (CDRs) of an antibody are the portions of the antibody which are largely responsible for antibody specificity. The CDR's directly interact with the epitope of the antigen (see, in general, Clark, 1986; Roitt, 1991). In both the heavy chain and the light chain variable regions of IgG immunoglobulins, there are four framework regions (FR1 through FR4) separated respectively by three complementarity determining regions (CDR1 through CDR3). The framework regions (FRs) maintain the tertiary structure of the paratope, which is the portion of the antibody which is involved in the interaction with the antigen. The CDRs, and in particular the CDR3 regions, and more particularly the heavy chain CDR3 contribute to antibody specificity. Because these CDR regions and in particular the CDR3 region confer antigen specificity on the antibody these regions may be incorporated into other antibodies or peptides to confer the identical specificity onto that antibody or peptide.

According to one embodiment, the peptide of the invention is an intact soluble monoclonal antibody in an isolated form or in a pharmaceutical preparation. An intact soluble monoclonal antibody, as is well known in the art, is an assembly of polypeptide chains linked by disulfide bridges. Two principle polypeptide chains, referred to as the light chain and heavy chain, make up all major structural classes (isotypes) of antibody. Both heavy chains and light chains are further divided into subregions referred to as variable regions and constant regions. As used herein the term “monoclonal antibody” refers to a homogenous population of immunoglobulins which specifically bind to art epitope (i.e. antigenic determinant).

The peptide useful according to the methods of the present invention may be an intact humanized a monoclonal antibody. A “humanized monoclonal antibody” as used herein is a human monoclonal antibody or functionally active fragment thereof having human constant regions and a binding CDR3 region from a mammal of a species other than a human. Humanized monoclonal antibodies may be made by any method known in the art. Humanized monoclonal antibodies, for example, may be constructed by replacing the non-CDR regions of a non-human mammalian antibody with similar regions of human antibodies while retaining the epitopic specificity of the original antibody. For example, non-human CDRs and optionally some of the framework regions may be covalently joined to human FR and/or Fc/pFc′ regions to produce a functional antibody. There are entities in the United States which will synthesize humanized antibodies from specific murine antibody regions commercially, such as Protein Design Labs (Mountain View Calif.).

European Patent Application 0239400, the entire contents of which is hereby incorporated by reference, provides an exemplary teaching of the production and use of humanized monoclonal antibodies in which at least the CDR portion of a murine (or other non-human mammal) antibody is included in the humanized antibody. Briefly, the following methods are useful for constructing a humanized CDR monoclonal antibody including at least a portion of a mouse CDR. A first replicable expression vector including a suitable promoter operably linked to a DNA sequence encoding at least a variable domain of an Ig heavy or light chain and the variable domain comprising framework regions from a human antibody and a CDR region of a murine antibody is prepared. Optionally a second replicable expression vector is prepared which includes a suitable promoter operably linked to a DNA sequence encoding at least the variable domain of a complementary human Ig light or heavy chain respectively. A cell line is then transformed with the vectors. Preferably the cell line is an immortalized mammalian cell line of lymphoid origin, such as a myeloma, hybridoma, trioma, or quadroma cell line, or is a normal lymphoid cell which has been immortalized by transformation with a virus. The transformed cell line is then cultured under conditions known to those of skill in the art to produce the humanized antibody.

As set forth in European Patent Application 0239400, several techniques are well known in the art for creating the particular antibody domains to be inserted into the replicable vector. (Preferred vectors and recombinant techniques are discussed in greater detail below.) For example, the DNA sequence encoding the domain may be prepared by oligonucleotide synthesis. Alternatively a synthetic gene lacking the CDR regions in which four framework regions are fused together with suitable restriction sites at the junctions, such that double stranded synthetic or restricted subcloned CDR cassettes with sticky ends could be ligated at the junctions of the framework regions. Another method involves the preparation of the DNA sequence encoding the variable CDR containing domain by oligonucleotide site-directed mutagenesis. Each of these methods is well known in the art. Therefore, those skilled in the art may construct humanized antibodies containing a murine CDR region without destroying the specificity of the antibody for its epitope.

Human monoclonal antibodies may be made by any of the methods known in the art, such as those disclosed in U.S. Pat. No. 5,567,610, issued to Borrebaeck et al., U.S. Pat. No. 565,354, issued to Ostberg, U.S. Pat. No. 5,571,893, issued to Baker et al, Kozber, J. Immunol. 133: 3001 (1984), Brodeur, et al., Monoclonal Antibody Production Techniques and Applications, p. 51-63 (Marcel Dekker, Inc, new York, 1987), and Boerner et al., J. Immunol., 147: 86-95 (1991). In addition to the conventional methods for preparing human monoclonal antibodies, such antibodies may also be prepared by immunizing transgenic animals that are capable of producing human antibodies (e.g., Jakobovits et al., PNAS USA, 90: 2551 (1993), Jakobovits et al., Nature, 362: 255-258 (1993), Bruggermann et al., Year in Immuno., 7:33 (1993) and U.S. Pat. No. 5,569,825 issued to Lonberg).

The binding peptides may also be functionally active antibody fragments. As is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York, Roitt, L (1991) Essential Immunology, 7th Ed., Blackwell Scientific Publications, Oxford). The pFc′ and Fc regions of the antibody, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. An isolated F(ab′)₂ fragment is referred to as a bivalent monoclonal fragment because of its two antigen binding sites. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd (heavy chain variable region). The Fd fragments are the major determinant of antibody specificity (a single Fd fragment may be associated with up to ten different light chains without altering antibody specificity) and Fd fragments retain epitope-binding ability in isolation.

The terms Fab, Fc, pFc′, F(ab′)₂ and Fv are used consistently with their standard immunological meanings [Klein, Immunology (John Wiley, New York, N.Y., 1982); Clark, W. R. (1986) The Experimental Foundations of Modern Immunology (Wiley & Sons, Inc., New York); Roitt, I. (1991) Essential Immunology, 7th Ed., (Blackwell Scientific Publications, Oxford)].

The invention also encompasses the use of single chain variable region fragments (scFv). Single chain variable region fragments are made by linking light and/or heavy chain variable regions by using a short linking peptide. Any peptide having sufficient flexibility and length can be used as a linker in a scFv. Usually the linker is selected to have little to no immunogenicity. An example of a linking peptide is multiple GGGGS [SEQ ID NO: 1] residues, which bridge the carboxy terminus of one variable region and the amino terminus of another variable region. Other linker sequences may also be used.

All or any portion of the heavy or light chain can be used in any combination. Typically, the entire variable regions are included in the scFv. For instance, the light chain variable region can be linked to the heavy chain variable region. Alternatively, a portion of the light chain variable region can be linked to the heavy chain variable region, or portion thereof. Also contemplated are scFvs in which the heavy chain variable region is from the antibody of interest, and the light chain variable region is from another immunoglobulin.

The scFvs can be assembled in any order, for example, V_{H}-linker-V_{L} or V_{L}-linker-V_{H}. There may be a difference in the level of expression of these two configurations in particular expression systems, in which case one of these forms may be preferred. Tandem scFvs can also be made, such as (X)-linker-(X)-linker-(X), in which X are polypeptides form the antibodies of interest, or combinations of these polypeptides with other polypeptides. In another embodiment, single chain antibody polypeptides have no linker polypeptide, or just a short, inflexible linker. Possible configurations are V_{L}-V_{H} and V_{H}-V_{L}. The linkage is too short to permit interaction between V_{L} and V_{H} within the chain, and the chains form homodimers with a V_{L}/V_{H} antigen binding site at each end. Such molecules are referred to in the art as “diabodies”.

Single chain variable regions may be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli, and the expressed protein may be isolated using standard protein purification techniques.

Conditions of expression should be such that the scFv polypeptide can assume optimal tertiary structure. Depending on the plasmid used and the host cell, it may be necessary to modulate the rate of production. For instance, use of a weaker promoter, or expression at lower temperatures, may be necessary to optimize production of properly folded scFv in prokaryotic systems; or it may be preferably to express scFv in eukaryotic cells.

Antibodies to M-CSF are known in the art. Non-limiting examples of commercially available anti-M-CSF antibodies include: Goat Anti-MCSF Polyclonal Antibody, Unconjugated (Abcam); Mouse Anti-MCSF Monoclonal Antibody, Unconjugated, Clone 116 (Abcam); Mouse Anti-MCSF Monoclonal Antibody, Unconjugated, Clone 21 (Abcam); Rabbit Anti-MCSF Polyclonal Antibody, Unconjugated (Abcam); Goat Anti-Human MCSF Antibody, Unconjugated (ABR-Affinity BioReagents); Mouse Anti-Human MCSF Monoclonal Antibody, Unconjugated, Clone 692 (ABR-Affinity BioReagents); Anti-Human M-CSF Antibody, Unconjugated (Antigenix America Inc.); Anti-Human M-CSF Polyclonal Antibody, Biotin Conjugated (Antigenix America Inc.); Anti-Human M-CSF Polyclonal Antibody, Unconjugated (Antigenix America Inc.); Anti-Mouse M-CSF Polyclonal Antibody, Biotin Conjugated (Antigenix America Inc.); Anti-Mouse M-CSF Polyclonal Antibody, Unconjugated (Antigenix America Inc.); Rat Anti-CSF-1 Monoclonal Antibody, Biotin Conjugated, Clone D24 (BD Biosciences Pharmingen); Rabbit Anti-Human M-CSF Polyclonal Antibody, Unconjugated (BIODESIGN International); Goat Anti-Mouse M-CSF (JE/MCP-1) Antibody, Biotin Conjugated (CEDARLANE Laboratories Limited); Goat Anti-Mouse M-CSF (JE/MCP-1) Antibody, Unconjugated (CEDARLANE Laboratories Limited); Anti-Human M-CSF Polyclonal Antibody, Biotin Conjugated (Cell Sciences); Anti-Human M-CSF Polyclonal Antibody, Unconjugated (Cell Sciences); Anti-Macrophage-Colony Stimulating Factor (M-CSF) Polyclonal Antibody, Unconjugated (CHEMICON); Goat Anti-MCSF Polyclonal Antibody, Unconjugated (GeneTex); Mouse Anti-MCSF Monoclonal Antibody, Unconjugated, 116 (GeneTex); Mouse Anti-MCSF Monoclonal Antibody, Unconjugated, 21 (GeneTex); Mouse Anti-MCSF Monoclonal Antibody, Unconjugated, 692 (GeneTex); Rabbit Anti-MCSF Polyclonal Antibody, Unconjugated (GeneTex); Mouse Anti-M-CSF Monoclonal Antibody, Unconjugated, Clone A00184.01 (GenScript); Goat Anti-Mouse M-CSF Polyclonal Antibody, Unconjugated (MBL International); Rabbit Anti-Human M-CSF Polyclonal Antibody, Unconjugated (MBL International); Goat Anti-MCSF Polyclonal Antibody, Unconjugated (Novus Biologicals); Mouse Anti-MCSF Monoclonal Antibody, Unconjugated, Clone 21 (Novus Biologicals); Mouse Anti-MCSF Monoclonal Antibody, Unconjugated, Clone 692 (Novus Biologicals); Mouse Anti-Human CSF1 Monoclonal Antibody, Unconjugated, Clone 1A9 (Novus Biologicals); Rabbit Anti-MCSF Polyclonal Antibody, Biotin Conjugated (Novus Biologicals); Rabbit Anti-MCSF Polyclonal Antibody, Unconjugated (Novus Biologicals); Goat Anti-Mouse M-CSF (Macrophage Colony Stimulating Factor) Polyclonal Antibody, Biotin Conjugated (PeproTech); Goat Anti-Mouse M-CSF (Macrophage Colony Stimulating Factor) Polyclonal Antibody, Unconjugated (PeproTech); Rabbit Anti-Human M-CSF (Macrophage Colony Stimulating Factor) Polyclonal Antibody, Biotin Conjugated (PeproTech); Rabbit Anti-Human M-CSF (Macrophage Colony Stimulating Factor) Polyclonal Antibody, Unconjugated (PeproTech); Goat Anti-Human M-CSF Polyclonal Antibody, Biotin Conjugated (R&D Systems); Goat Anti-Human M-CSF Polyclonal Antibody, Unconjugated (R&D Systems); Goat Anti-Mouse M-CSF Polyclonal Antibody, Biotin Conjugated (R&D Systems); Goat Anti-Mouse M-CSF Polyclonal Antibody, Unconjugated (R&D Systems); Mouse Anti-Human M-CSF Monoclonal Antibody, Allophycocyanin Conjugated, Clone 26786 (R&D Systems); Mouse Anti-Human M-CSF Monoclonal Antibody, Phycoerythrin Conjugated, Clone 26786 (R&D Systems); Goat Anti-MCSF Polyclonal Antibody, Unconjugated (Abcam); Mouse Anti-MCSF Monoclonal Antibody, Unconjugated, Clone 116 (Abcam); Mouse Anti-MCSF Monoclonal Antibody, Unconjugated, Clone 21 (Abcam); Mouse Anti-MCSF Monoclonal Antibody, Unconjugated, Clone 692 (Abcam); Rabbit Anti-MCSF Polyclonal Antibody, Unconjugated (Abcam); Goat Anti-Human MCSF Antibody, Unconjugated (ABR-Affinity BioReagents); Goat Anti-Mouse MCSF Antibody, Unconjugated (ABR-Affinity BioReagents); Mouse Anti-Human MCSF Monoclonal Antibody, Unconjugated, Clone 692 (ABR-Affinity BioReagents); Goat Anti-MCSF Polyclonal Antibody, Unconjugated (GeneTex); Mouse Anti-MCSF Monoclonal Antibody, Unconjugated, 116 (GeneTex); Mouse Anti-MCSF Monoclonal Antibody, Unconjugated, 21 (GeneTex); Mouse Anti-MCSF Monoclonal Antibody, Unconjugated, 692 (GeneTex); Rabbit Anti-MCSF Polyclonal Antibody, Unconjugated (GeneTex); Goat Anti-MCSF Polyclonal Antibody, Unconjugated (Novus Biologicals); Mouse Anti-MCSF Monoclonal Antibody, Unconjugated, Clone 21 (Novus Biologicals); Mouse Anti-MCSF Monoclonal Antibody, Unconjugated, Clone 692 (Novus Biologicals); Mouse Anti-Human CSF1 Monoclonal Antibody, Unconjugated, Clone 1A9 (Novus Biologicals); Mouse Anti-Human CSF1 Polyclonal Antibody, Unconjugated (Novus Biologicals); Rabbit Anti-MCSF Polyclonal Antibody, Biotin Conjugated (Novus Biologicals); and, Rabbit Anti-MCSF Polyclonal Antibody, Unconjugated (Novus Biologicals). The antibodies may be used or modified as described herein.

Antagonistic effects (e.g., down-regulation) for M-CSF may be achieved by the use of M-CSF expression inhibitors, including agents which interfere with M-CSF expression at either the mRNA or protein level. Such inhibitors are referred to as expression inhibitors or M-CSF expression inhibitors. Examples of expression inhibitors include, for instance, antisense oligonucleotides and therapeutic RNA.

Small interfering RNA (siRNA), sometimes known as short interfering RNA or silencing RNA, are a class of short (generally 20-25 nucleotide-long) double-stranded RNA molecules that play a variety of roles in biology. Most notably, siRNA is involved in the RNA interference (RNAi) pathway where the siRNA interferes with the expression of a specific gene. In addition to their role in the RNAi pathway, siRNAs also act in RNAi-related pathways, e.g. as an antiviral mechanism or in shaping the chromatin structure of a genome.

Thus, the methods of the invention also encompass use of isolated short RNA that directs the sequence-specific degradation of a M-CSF mRNA through a process known as RNA interference (RNAi). The process is known to occur in a wide variety of organisms, including embryos of mammals and other vertebrates. It has been demonstrated that dsRNA is processed to RNA segments 21-23 nucleotides (nt) in length, and furthermore, that they mediate RNA interference in the absence of longer dsRNA. Thus, these fragments are sequence-specific mediators of RNA degradation and are referred to herein as siRNA or RNAi. Methods of the invention encompass the use of these fragments (or recombinantly produced or chemically synthesized oligonucleotides of the same or similar nature) to enable the targeting of M-CSF mRNAs for degradation in mammalian cells useful in the therapeutic applications discussed herein.

The methods for design of the RNA's that mediate RNAi and the methods for transfection of the RNAs into cells and animals is well known in the art and the RNAi molecules are readily commercially available (Verma N. K. et al, J. Clin. Pharm. Ther., 28(5):395-404 (2004), Mello C. C. et al. Nature, 431(7006)338-42 (2004), Dykxhoorn D. M. et al., Nat. Rev. Mol. Cell. Biol. 4(6):457-67 (2003) Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK)). The RNAs are preferably chemically synthesized using appropriately protected ribonucleoside phosphoramidites and a conventional DNA/RNA synthesizer. Most conveniently, siRNAs are obtained from commercial RNA oligo synthesis suppliers listed herein. In general, RNAs are not too difficult to synthesize and are readily provided in a quality suitable for RNAi. A typical 0.2 μmol-scale RNA synthesis provides about 1 milligram of RNA, which is sufficient for 1000 transfection experiments using a 24-well tissue culture plate format.

The M-CSF cDNA specific siRNA is designed preferably by selecting a sequence that is not within 50-100 bp of the start codon and the termination codon, avoids intron regions, avoids stretches of 4 or more bases, such as AAAA, CCCC, avoids regions with GC content of <30% or >60%, avoids repeats and low complex sequence, and it avoids single nucleotide polymorphism sites. The target sequence may have a GC content of around 50%. The siRNA targeted sequence may be further evaluated using a BLAST homology search to avoid off target effects on other genes or sequences. Negative controls are designed by scrambling targeted siRNA sequences. The control RNA preferably has the same length and nucleotide composition as the siRNA but has at least 4-5 bases mismatched to the siRNA. The RNA molecules of the present invention can comprise a 3′ hydroxyl group. The RNA molecules can be single-stranded or double stranded; such molecules can be blunt ended or comprise overhanging ends (e.g., 5′, 3′) from about 1 to about 6 nucleotides in length (e.g., pyrimidine nucleotides, purine nucleotides). In order to further enhance the stability of the RNA of the present invention, the 3′ overhangs can be stabilized against degradation. The RNA can be stabilized by including purine nucleotides, such as adenosine or guanosine nucleotides. Alternatively, substitution of pyrimidine nucleotides by modified analogues, e.g., substitution of uridine 2 nucleotide 3′ overhangs by 2′-deoxythymidine is tolerated and does not affect the efficiency of RNAi. The absence of a 2′ hydroxyl significantly enhances the nuclease resistance of the overhang in tissue culture medium.

The RNA molecules used in the methods of the present invention can be obtained using a number of techniques known to those of skill in the art. For example, the RNA can be chemically synthesized or recombinantly produced using methods known in the art. Such methods are described in U.S. Published Patent Application Nos. US2002-0086356A1 and US2003-0206884A1 that are hereby incorporated by reference in their entirety.

Any RNA can be used in the methods of the present invention, provided that it has sufficient homology to the M-CSF gene to mediate RNAi. The RNA for use in the present invention can correspond to the entire M-CSF gene or a portion thereof. There is no upper limit on the length of the RNA that can be used. For example, the RNA can range from about 21 base pairs (bp) of the gene to the full length of the gene or more. In one embodiment, the RNA used in the methods of the present invention is about 1000 bp in length. In another embodiment, the RNA is about 500 bp in length. In yet another embodiment, the RNA is about 22 bp in length. In certain embodiments the preferred length of the RNA of the invention is 21 to 23 nucleotides. The Sequence of M-CSF is known, and thus those skilled in the art can readily use the embodiments disclosed herein.

The invention also embraces antisense oligonucleotides that selectively bind to a nucleic acid molecules encoding M-CSF to decrease expression and activity of this protein and subunits thereof. Antisense oligonucleotides can be designed to interfere with expression of the M-CSF based on the known nucleotide sequence of the M-CSF.

As used herein, the term “antisense oligonucleotide” or “antisense” describes an oligonucleotide that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA. The antisense oligonucleotide molecules are designed so as to interfere with transcription or translation of a target gene upon hybridization with the target gene or transcript. Antisense oligonucleotides that selectively bind to a nucleic acid molecule encoding M-CSF are particularly preferred. Those skilled in the art will recognize that the exact length of the antisense oligonucleotide and its degree of complementarity with its target will depend upon the specific target selected, including the sequence of the target and the particular bases which comprise that sequence.

It is preferred that the antisense oligonucleotide be constructed and arranged so as to bind selectively with the target under physiological conditions, i.e., to hybridize substantially more to the target sequence than to any other sequence in the target cell under physiological conditions. Based upon the nucleotide sequences of nucleic acid molecules encoding M-CSF or upon allelic or homologous genomic and/or cDNA sequences, one of skill in the art can easily choose and synthesize any of a number of appropriate antisense oligonucleotide molecules for use in accordance with the present invention. In order to be sufficiently selective and potent for inhibition, such antisense oligonucleotides should comprise at least about 10 and, more preferably, at least about 15 consecutive bases which are complementary to the target, although in certain cases modified oligonucleotides as short as 7 bases in length have been used successfully as antisense oligonucleotides. See Wagner et al., Nat. Med. 1(11):1116-1118, 1995. Most preferably, the antisense oligonucleotides comprise a complementary sequence of 20-30 bases. Although oligonucleotides may be chosen which are antisense to any region of the gene or mRNA transcripts, in preferred embodiments the antisense oligonucleotides correspond to N-terminal or 5′ upstream sites such as translation initiation, transcription initiation or promoter sites. In addition, 3′-untranslated regions may be targeted by antisense oligonucleotides. Targeting to mRNA splice sites has also been used in the art but may be less preferred if alternative mRNA splicing occurs. In addition, the antisense is targeted, preferably, to sites in which mRNA secondary structure is not expected (see, e.g., Sainio et al., Cell Mol. Neurobiol. 14(5):439-457, 1994) and at which proteins are not expected to bind.

In one set of embodiments, the antisense oligonucleotides of the invention may be composed of “natural” deoxyribonucleotides, ribonucleotides, or any combination thereof. That is, the 5′ end of one native nucleotide and the 3′ end of another native nucleotide may be covalently linked, as in natural systems, via a phosphodiester internucleoside linkage. These oligonucleotides may be prepared by art recognized methods which may be carried out manually or by an automated synthesizer. They also may be produced recombinantly by vectors.

In preferred embodiments, however, the antisense oligonucleotides of the invention also may include “modified” oligonucleotides. That is, the oligonucleotides may be modified in a number of ways which do not prevent them from hybridizing to their target but which enhance their stability or targeting or which otherwise enhance their therapeutic effectiveness.

The term “modified oligonucleotide” as used herein describes an oligonucleotide in which (1) at least two of its nucleotides are covalently linked via a synthetic internucleoside linkage (i.e., a linkage other than a phosphodiester linkage between the 5′ end of one nucleotide and the 3′ end of another nucleotide) and/or (2) a chemical group not normally associated with nucleic acid molecules has been covalently attached to the oligonucleotide. Preferred synthetic internucleoside linkages are phosphorothioates, alkylphosphonates, phosphorodithioates, phosphate esters, alkylphosphonothioates, phosphoramidates, carbamates, carbonates, phosphate triesters, acetamidates, carboxymethyl esters and peptides.

The term “modified oligonucleotide” also encompasses oligonucleotides with a covalently modified base and/or sugar. For example, modified oligonucleotides include oligonucleotides having backbone sugars which are covalently attached to low molecular weight organic groups other than a hydroxyl group at the 3′ position and other than a phosphate group at the 5′ position. Thus modified oligonucleotides may include a 2′-O-alkylated ribose group. In addition, modified oligonucleotides may include sugars such as arabinose instead of ribose.

The present invention, thus, contemplates pharmaceutical preparations containing modified antisense oligonucleotide molecules that are complementary to and hybridizable with, under physiological conditions, nucleic acid molecules encoding M-CSF, together with pharmaceutically acceptable carriers. Antisense oligonucleotides may be administered as part of a pharmaceutical composition. Such a pharmaceutical composition may include the antisense oligonucleotides in combination with any standard physiologically and/or pharmaceutically acceptable carriers which are known in the art. The compositions should be sterile and contain a therapeutically effective amount of the antisense oligonucleotides in a unit of weight or volume suitable for administration to a subject.

TRAIL receptor activators also include agonists for TRAIL. An “agonist” is a molecule that selectively binds to a specific receptor and triggers a response in the cell. It mimics the action of an endogenous biochemical molecule that binds to the same receptor. As used herein, the term “TRAIL receptor agonist” refers to a molecule which binds to and activates a TRAIL receptor.

TRAIL, which stands for “TNF-related apoptosis-inducing ligand” is a ligand molecule which induces the process of cell death called apoptosis. It is a type II transmembrane protein with homology to other members of the tumor necrosis factor family. In humans, the gene that encodes for TRAIL is located at chromosome 3q26. TRAIL binds to the death receptors, DR4 and DR5. The process of apoptosis is thought to be caspace-dependent.

Death receptors are cell surface receptors that transmit apoptosis signals initiated by specific ligands. They play an important role in apoptosis and can activate a caspase cascade within seconds of ligand binding. Induction of apoptosis via this mechanism is therefore very rapid. Death receptors belong to the tumour necrosis factor (TNF) gene superfamily and generally can have several functions other than initiating apoptosis.

The apoptotic signals by these cytokines are transduced by eight different death domain-(DD) containing receptors (TNFR1, also called DR1; Fas, also called DR2; DR3, DR4, DR5, DR6, NGFR, and EDAR). The intracellular portion of all these receptors contains a region approximately 80 amino acids long referred to as the “death domain.” Upon activation by its ligand, the DD recruits various proteins that mediate both death and proliferation of the cells. These proteins in turn recruit other proteins via their DDs or death effector domains. The death domain (DD) forms a highly compact structure comprising six amphipathic, antiparallel, α-helices (Huang, B., Eberstadt, M., Olejniczak, E. T., Meadows, R. P. and Fesik, S. W., (1996) Nature, 384, 638-641; Weber, C. H. and Vincenz, C. (2001) FEBS Lett., 492, 171-176).

In some embodiments, activating antibodies may be employed to specifically bind to and act on the TRAIL receptors that are present on cell surface, whereby functioning as a receptor ligand to activate the death receptor or death receptors.

It is also possible in some cases to alter the efficacy of death receptors, for instance, by manipulating the status and the corresponding activity levels of such receptors, via, for instance, post-translational modification including phosphorylation and internalization.

The invention further provides in one aspect methods for treating a subject having an HIV infection by selectively inducing TRAIL-mediated apoptosis in an HIV-infected cell. The methods involve administration of a TRAIL receptor activator. In certain embodiments, the TRAIL receptor activator may be a nucleic acid construct encoding at least a portion of a TRAIL receptor gene sequence. Typically, the gene sequence is introduced into cells as an expression construct, such as a vector plasmid which contains such a nucleic acid sequence.

In certain embodiments, TRAIL receptor expression may be enhanced by the means of gene transfer, e.g., transfection and infection of reservoir cell with a construct encoding such a receptor. The cDNA sequence of TRAIL receptors is known in the art. In general, such cDNA sequence may be inserted into a suitable expression vector, such as a plasmid, linked to an appropriate promoter system to allow transcription and translation of the gene or gene fragment(s). Such strategies entailing recombinant molecular biological techniques are routinely used in the art. In some cases, it is desirable to select a promoter system that preferentially becomes activated in a cell-type-specific or tissue-specific manner.

In some embodiments, an inhibitor of topoisomerase II may be used to activate or enhance the action of the TRAIL receptor. A number of topoisomerase II inhibitors are known and available in the art. Non-limiting examples of topoisomerase II inhibitors include: Etoposide (Eposin®, Etopophos®, Vepesid®, VP-16® and teniposide.

The invention further contemplates that the methods for inducing apoptosis in HIV reservoir cells may be enhanced by concomitant activation of TRAIL receptor signaling. The activation of TRAIL receptor signaling may be achieved by a number of ways, including contacting the TRAIL receptor-expressing cell with a TRAIL molecule or agonists thereof, which act as a ligand for the TRAIL receptor. Such TRAIL molecule or agonists may be in a soluble form or expressed on the cell surface of a neighboring cell, such that TRAIL receptor activation may occur in trans by cell-cell interaction. For example, lymphocytes, such as NK cells, CD4+ T cells and CD8+ T cells, express TRAIL on the cell surface, which then can bind to and activate TRAIL receptors that are expressed on a reservoir cell. Therefore, inducing the expression of TRAIL ligand may promote activation of TRAIL receptor signaling in the reservoir cell.

The invention provides, in further aspects, methods for upregulating cell surface receptors that mediate apoptosis in HIV reservoir cells. More specifically, the methods are drawn to increasing expression of so-called death receptors, DR4 and DR5 in particular, by using an M-CSF antagonist to the reservoir cell. Based on the recognition that HIV can cause M-CSF-dependent change in TRAIL receptor expression, the invention thus provides methods for counteracting such effects. This may be achieved by, for example, contacting the HIV-infected reservoir cell with an effective amount of an M-CSF antagonist to upregulate cell surface death receptors, and contacting the cell with TRAIL ligand or an agonist thereof.

Thus, a number of means are contemplated to antagonize M-CSF action. For example, in some cases, an antibody that specifically bind to M-CSF ligand and neutralize its effects may be used. Similarly, the siRNA approach may be used to silence the M-CSF gene. In addition, M-CSF effects may be suppressed by inhibiting the receptor kinase activity by using, for example, Gleevec® Imatinib mesylate; ST1571); Nilotinib; Dasatinib; Sorafenib; Sunitinib; GW2580, 5-{3-methoxy-4-[(4methoxybenzyl)oxy]benzyl}pyrimidine-2,4-diamine; ABT-869; AG013736; BAY 43-9006; CHIR258; SU11248.

According to another aspect of the invention, methods for treating a subject having an HIV infection are provided. The subject is administered a TRAIL receptor activator, such as an M-CSF effector kinase inhibitor, that effectively inhibits M-CSF signaling, without substantially affecting HIV replication per se. The methods of the invention are particularly useful for the treatment of a Human immunodeficiency virus (HIV) infection. HIV, a species of retrovirus also known as human T-cell lymphotropic virus III (HTLV III), is responsible for causing the deterioration resulting in the disorder known as AIDS. HIV infects and destroys T-cells, upsetting the overall balance of the immune system, resulting in a loss in the patients ability to combat other infections and predisposing the patient to opportunistic infections which frequently prove fatal.

More specifically, the invention includes methods for treating a subject having an HIV infection by administering an M-CSF effector kinase inhibitor that can effectively induce TRAIL-mediated apoptosis in HIV reservoir cells and cause a reduction in a number of such cells in the infected subject, without directly affecting the replication process of HIV.

Thus, the invention includes the surprising finding that M-CSF inhibition and its potential therapeutic effects for HIV treatment did not directly interfere with HIV replication per se. Instead, the effect of M-CSF inhibition is at least primarily mediated by its ability to selectively induce cell death in HIV-infected reservoir cells. Accordingly, in some embodiments, the invention provides methods for inducing apoptosis in an HIV-infected reservoir cell by an inhibitor of M-CSF signaling, which does not directly affect HIV viral replication.

The invention, therefore, provides compositions and methods for inducing apoptosis in an HIV reservoir cell by inhibiting M-CSF signaling pathway. In particular, the methods relate to targeting HIV reservoir cells with a kinase inhibitor that inhibits an M-CSF effector. Accordingly, the compositions and methods disclosed herein may be useful for combating viruses that persist in a pool of reservoir cells that are relatively resistant to other anti-retroviral therapy, such as Highly Active Anti-Retroviral Therapy, or HAART.

As used herein, “inducing apoptosis” refers to a specific signal or signals, such as ligand binding to its cell surface receptor and the subsequent activation of the receptor, that trigger a cascade of signaling events leading to programmed cell death. The process is thought to involve caspase-dependent pathways.

The term “a reservoir cell” refers in general to an infected cell containing integrated proviruses that are in some cases transcriptionally dormant, or latent. Thus, in a purely latent reservoir cell infected with HIV, viral gene expression does not occur, or is significantly limited, until the cellular host later becomes activated. However, “a reservoir cell’ as used herein shall broadly encompass those cells that are not technically latent (i.e., transcriptionally dormant), but are capable of supporting provirus that can ultimately lead to secondary infection or dissemination. Accordingly, “an HIV reservoir cell” shall refer to such an infected cell that can act as a reserved source of HIV virions that can lead to subsequent secondary infection upon activation. Non-limiting examples of an HIV reservoir cell include: macrophages, memory CD4+ T cells, monocytes and dendritic cells. Macrophages are non-dividing cells, which, when infected with HIV-1 may function as a reservoir, yet permissive for all steps of the viral life cycle.

Some embodiments of the invention are drawn to methods of inducing apoptosis in HIV reservoir cells using an agent that inhibits M-CSF signaling pathway, in conjunction with an agent that inhibits histone deacetylase (HDAC) activity. Histone deacetylases are a class of enzymes that remove acetyl groups from an ε-N-acetyl lysine amino acid on a histone. Deacetylation restores the positive electric charge of the lysine amino acids, which increases the histone's affinity for the negatively charged phosphate backbone of DNA. They are associated with the formation of heterochromatin. This generally down-regulates DNA transcription by blocking the access of transcription factors. HDAC inhibitors have been shown to regulate the expression of certain tumor suppressor activity. For this reason, HDAC inhibitos have been studied as a treatment for cancer. HDAC inhibitors are also associated with the downregulation of some gene promoters. This could be due to upregulation of other, negative-regulatory proteins. As described elsewhere herein in more detail, the invention includes the finding that inhibiting the cell protective effect of HIV-1 envelope to induce apoptosis in the HIV-infected reservoir is augmented in the presence of HDAC inhibitor.

In further embodiments, the invention contemplates methods for treating a subject having an HIV infection, drawn to administering a combination of a histone deacetylase inhibitor and a TRAIL receptor activator.

Naturally occurring inhibitors of HDAC include, but are not limited to: trapoxin B (TPX); trichostatin A (TSA); chlamydocin; and suberoyl anilide hydroxamic acid (SAHA). Other HDAC inhibitors that are commercially available include, but are not limited to: APHA Compound 8; Apicidin; Sodium Butyrate; (−)-Depudecin; criptaid; and Sirtinol.

The HDAC inhibitors can be classified into at least five sub-categories based on their chemical compositions. HDAC inhibitors that are based on short-chain fatty acids include: Butyrate; phenylbutyrate; and Valproate. HDAC inhibitors that are based on hydroxamic acids include: the trichostatins; SAHA and its derivatives; Oxamflatin; ABHA; Scriptaid (SB-556629); Pyroxamide; Propenamides; and Aroyl pyrrolyl hydroxyamides. HDAC inhibitors that are based on amides include: MS-275 (MS-27-275); MethyGene; and CI-994. HDAC inhibitors based on epoxyketone-containing cyclic tetrapeptides include: the trapoxins; HC-toxin; Chlamydocin; Diheteropeptin; WF-3161; and Cy1-1 and Cy1-2. HDAC inhibitors that are non-epoxyketone-containing cyclic tetrapeptides include: FR901228 (FK228, depsipeptide); and the cyclic-hydroxamic-acid-containing peptides (CHAPs). Others include: Depudecin; Tubacin; and Organosulfur compounds.

There are a number of HDAC inhibitors which are currently in clinical development. Examples are listed below. Amongst the HDAC inhibitors that are in the Phase II clinical trials include: CI-994 (Pfizer); FK228 (Gloucester); SAHA (Merck & co); MS-275 (MS-27-275); Pivanex (Titan); and PXD11 (CuraGen, TopoTarget). Amongst the HDAC inhibitors that are in the Phase 1 clinical trials include: MGCD0103 (MethylGene); LBH589 (Novartis); and NVP-LAQ824 (Novartis).

In any of the above-mentioned embodiments, where a histone deacetylase inhibitor is used, one or more of such inhibitors may be used.

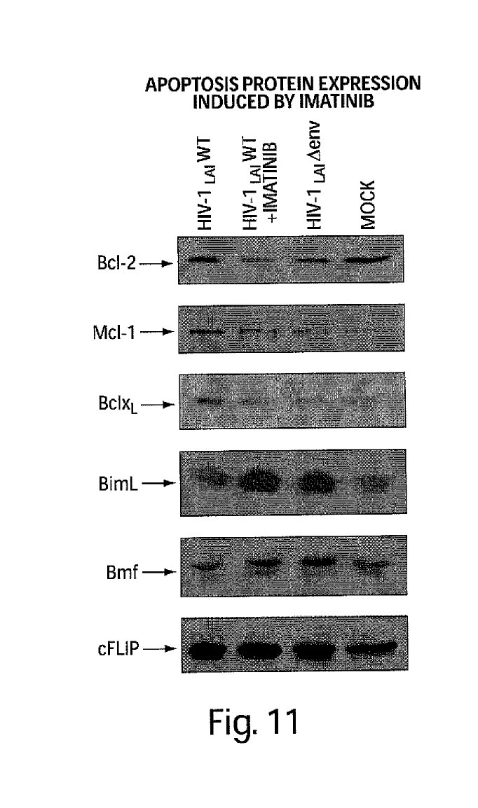

The invention contemplates methods for treating an HIV-infected subject using one or more agents that target apoptotic pathways, including but are not limited to: Death receptors, CD95/Fas, TNF, Caspases, IAPs/SMAC, Bcl-2 and p53. In some embodiments, such agent may be used alone or used in combination. In some embodiments, the invention contemplates methods for inducing apoptosis in HIV reservoir cells using an agent that inhibits M-CSF signaling pathway in conjunction with an agent that favors activation of apoptotic pathway(s). It has been reported that viral envelope can induce gene expression of anti-apoptotic genes, such as Mcl-1, IAPs and XIAP. FIG. 11, for example, provides data showing that a subset of genes known to be involved in cellular apoptotic regulations elicit increased protein expression in response to Imatinib treatment. A number of agents that favor activation of apoptotic pathway(s) are being developed and exploited for therapeutic use, cancer treatment in particular. For example, several classes of molecular targets have been considered as apoptosis-based therapeutics. These include, but are not limited to, reagents that target the following signaling molecules and/or expression thereof: Death receptors (such as TRAIL receptors), CD95/Fas, TNF, Caspases (such as pan-caspase, Caspase-1, Caspase-3, Caspase-6 and Caspase-9), IAPs/SMAC (Inhibitor of Apoptosis Protein/second mitochondrial activator of caspases), Bcl-2 (including anti-apoptotic Bcl-2 members and pro-apoptotic Bcl-2 members) and p53. Based on the finding as described in the instant invention, therefore, these agents may be useful for targeting HIV-infected reservoir cells and preferentially inducing apoptosis in these cells.

Accordingly, the invention provides methods for inducing apoptosis in HIV-infected reservoir cells using one or more agents directed to these molecular targets for regulating apoptosis, in combination with an agent that inhibits an M-CSF-dependent signaling pathway. A number of agents that target apoptotic pathways are thus contemplated for certain embodiments of this invention.

Non-limiting examples of agents that target death receptors, e.g., TRAIL receptor, include: HGS-ETR1, HGS-ERT2, HGS-TR2J (Human Genome Sciences) and PRO1762 (Amgen/Genentech). Non-limiting examples of agents that target IAPs/SMAC include: BIR3 antagonists (Idun), Capped tripeptide XIAP antagonists (Abbott; Oost et al.), TWX024 (Wu et al.), Polyphenylurea derivatives (Schimmer al., 2004), Smac-mimetic compounds (Sun et al., 2004), Embelin (Nikolovska-Coleska et al., 2004), XIAP antisense and RNAi constructs (Hu et al., 2003; Bilim et al., 2003; McManus et al., 2004), AGE35156/GEM®640 (Aegera/Hybridon), HIV-Tat- and polyarginine-conjugated SMAC peptides (Fulda et al., 2002; Yang et al., 2003), Nonpeptide small-molecule SMAC mimetic (Li et al., 2004), Benzenesulphonamide derivative (Novartis Genome Foundation), Tripeptides (Abbott Laboratories), Constrained peptidomimetic (University of Michigan), Embeline (University of Michigan), Di/triphenylureas (1396-11, 12, 34) (The Burnham Institute/TPIMS) and Compound 3 (University of Texas Southwestern Medical Center).

Non-limiting examples of agents that target Bcl-2 include: Bcl-2 blocker (Idun; Abott), GX01 series of compounds (Gemin X), Bcl-2 small-molecule antagonist (Structural Bioinformatics), Tetrocarcin-A derivatives (Kyowa Hakko Kogyo Co., Ltd.), Chelerythrine (Chan et al., 2003), Antimycin A derivatives (Wang and Liu et al., 2000), HA14-1 (Wang and Zhang et al., 2000), Synthetic compound binding to the BH3 of bcl-2 (Enyedy et al., 2001), Genasense (Aventis/Genta Inc.), Bispecific bcl-2/Bcl-xL antisense (Zangemeister-Wittke et al., 2000), BH3 peptides from Bax, Bak, Bid or Bad (Holinger et al., 1999; Wang et al., 2000), SAHBs (Walensky et al., 2004), BH3Is (Degterev et al., 2001; Harvard University), Gossypol (National Cancer Institute/Ascenta), Polyphenol E (Mayo (Mitsui Norin)), HA14-1 analogues/CPM-1285 analogues (Raylight Chemokine Pharmaceuticals), HB3I-1/BH31-2 (Harvard University), Antimycin A3 (University of Washington), Compound 6 (University of Michigan), Terphenyl derivative (Yale University), Apogossypol (Burnham/National Cancer Institute), A-779024 (ABT 737) (Abbott Laboratories) and 3C1-AHPC/MM11453 (Burnham).

It should be appreciated that one or more of the agents listed above, as well as any other agents that target one or more of apoptotic pathways exemplified above, may be used to induce apoptosis in HIV-infected reservoir cells. Such agent or agents may synergize the effect of an M-CSF inhibitor in inducing apoptosis of HIV-infected reservoir cells, such as macrophages.

As used herein, a subject is a human or a non-human animal that is susceptible to immunodeficiency virus infection, including, but not limited to a non-human primates. In some embodiments, human subjects are preferred.

The terms, as used herein, “administering” “administer” and “administration” refers to the process of applying, dispensing or making available the reagent or medicament to a subject. A number of modes of administration of a therapeutic agent are available and are known in the art.

In a subject infected with HIV or at risk of such infection, an effective amount of a TRAIL receptor activator. In some embodiments the effective amount of TRAIL receptor activator is not an effective amount for directly inhibiting HIV replication. For any compound described herein a therapeutically effective amount can be initially determined from cell culture assays. In particular, the effective amount of TRAIL receptor activator can be determined using in vitro assays. Effective amounts of a composition of the invention may also be determined by assessing physiological effects of administration on a cell or subject, such as a decrease in viral load following administration. Other assays will be known to one of ordinary skill in the art and can be employed for measuring the level of the response to a treatment. The amount of a treatment may be varied for example by increasing or decreasing the amount of a therapeutic composition, by changing the therapeutic composition administered, by changing the route of administration, by changing the dosage timing and so on. The effective amount will vary with the particular condition being treated, the age and physical condition of the subject being treated, the severity of the condition, the duration of the treatment, the nature of the concurrent therapy (if any), the specific route of administration, and the like factors within the knowledge and expertise of the health practitioner. For example, an effective amount may depend upon the stage and pathogenesis of the HIV infection and/or AIDS.

Therapeutically effective amounts can also be determined in animal studies. For instance, the effective amount of TRAIL receptor activator alone or in combination with anti-HIV therapy to induce a therapeutic response can be assessed using in vivo assays of viral load. Relevant animal models include primates infected with simian immunodeficiency virus (SIV). Generally, a range of TRAIL receptor activator doses are administered to the animal optionally along with a range of anti-HIV therapy doses. Reduction in viral load in the animals following the administration of the active agents is indicative of the ability to reduce the viral load and thus treat HIV infection.

Typically, an effective amount of a drug to induce apoptosis in HIV-infected reservoir cells will be determined in clinical trials, establishing an effective dose for a test population versus a control population in a blind study. In some embodiments, an effective amount will be that results in a desired response, e.g., an amount that effectively induces apoptosis in HIV-infected reservoir cells in the subject. Thus, an effective amount may be the amount that when administered reduces the viral load in the subject to an amount that that is above the amount that would occur in the subject or tissue without the administration of the composition.

The art is familiar with techniques used to determine viral load in a sample. Preferably, viral load is measured not as plasma RNA copies, indicative of HIV replication in the activated CD4+ T cell compartment, but by highly sensitive Q-PCR for HIV 2LTR circles that is indicative of new rounds of infection. This method is used to show ongoing viral replication in the face of HAART (See Sharkey et al., 2000).

The applied dose of the TRAIL receptor activator and optionally the anti-HIV therapy can be adjusted based on the relative bioavailability and potency of the administered compounds. Adjusting the dose to achieve maximal efficacy based on the methods described above and other methods are well within the capabilities of the ordinarily skilled artisan. Most of the anti-HIV therapies have been identified. These amounts can be adjusted when they are combined with TRAIL receptor activators by routine experimentation.

Subject doses of the compounds described herein typically range from about 0.1 μg to 10,000 mg, more typically from about 1 μg/day to 8000 mg, and most typically from about 10 μg to 100 μg. Stated in terms of subject body weight, typical dosages range from about 0.1 μg to 20 mg/kg/day, more typically from about 1 to 10 mg/kg/day, and most typically from about 1 to 5 mg/kg/day.

Effective amounts will also depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of an composition to induce apoptosis in HIV-infected reservoir cells (alone or in combination with other therapeutic agents) be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The absolute amount will depend upon a variety of factors, including the material selected for administration, whether the administration is in single or multiple doses, and individual subject parameters including age, physical condition, size, weight, and the stage of the disease or condition. These factors are well known to those of ordinary skill in the art and can be addressed with no more than routine experimentation.

In some cases, it may be desirable to target the pharmaceutical compound, at least as part of a regime, to a specific tissue or tissues. For example, Imatinib has poor bioavailability to the brain, which is a site of HIV infection of macrophages. Co-administration with hydroxyurea, which is also an anti-viral agent, may promote tissue availability.

Generally, the pharmaceutical compound used in the methods of the instant invention may be coupled to a vectorizing agent to facilitate bioavailability in a desired tissue or tissues. As used herein, the term “vectorized” refers to engineered moieties or modifications to a subject agent or compound for the purpose of delivering the composition to a target site in a cell or a tissue. For example, vectorized agents are produced by covalently linking a compound to a moiety which promotes delivery from the circulation to a predetermined destination in the body. In some examples, antibodies are linked to another macromolecule, the antibodies being the agent which promotes delivery of the macromolecules. One example of such an agent is an antibody which is directed towards a cell surface component, such as a receptor, which is transported away from the cell surface.

Pharmaceutical compounds of the invention may be administered alone, in combination with each other, and/or in combination with other drug therapies, or other treatment regimens that are administered to subjects with HIV infection. An anti-HIV therapy, as used herein is any therapeutic that is useful for reducing viral load, preventing viral infection, prolonging the asymptotic phase of HIV infection, or prolonging the life of a subject infected with HIV. Anti-HIV therapies include but are not limited to inhibitors of HIV replication, such as protease inhibitors, e.g., HAART; cytokines; and chemokines. Didanosine (2′-3′-dideoxyinosine, ddI) is sold under the trade names Videx® and Videx EC®. It is a reverse transcriptase inhibitor, effective against HIV and used in combination with other antiretroviral drug therapy as part of highly active antiretroviral therapy (HAART). Didanosine (ddI) is a nucleoside analogue of adenosine having hypoxanthine attached to the sugar ring. Emtricitabine (FTC), with trade name Emtriva® (formerly Coviracil), is a nucleoside reverse transcriptase inhibitor (NRTI) for the treatment of HIV infection in adults. Emtricitabine is an analogue of cytidine. Enfuvirtide (INN) is an HIV fusion inhibitor, marketed under the trade name Fuzeon (Roche). Nevirapine, also marketed under the trade name Viramune® (Boehringer Ingelheim), is a non-nucleoside reverse transcriptase inhibitor (NNRTI) used to treat HIV-1 infection and AIDS but is a protease inhibitor. Stavudine (2′-3′-didehydro-2′-3′-dideoxythymidine, d4T, brand name Zerit®) is a nucleoside analog reverse transcriptase inhibitor (NARTI) active against HIV. Stavudine is an analog of thymidine.

In some embodiments of the invention, the TRAIL receptor activator and the anti-HIV therapy may be administered at the same time or in alternating cycles or any other therapeutically effective schedule. “Alternating cycles” as used herein, refers to the administration of the different active agents at different time points. The administration of the different active agents may overlap in time or may be temporally distinct. The cycles may encompass periods of time which are identical or which differ in length. For instance, the cycles may involve administration of the TRAIL receptor activator on a daily basis, every two days, every three days, every four days, every five days, every six days, a weekly basis, a monthly basis or any set number of days or weeks there-between, every two months, three months, four months, five months, six months, seven months, eight months, nine months, ten months, eleven months, twelve months, etc, with the anti-HIV therapy being administered in between. Alternatively, the cycles may involve administration of the TRAIL receptor activator on a daily basis for the first week, followed by a monthly basis for several months, and then every three months after that, with the anti-HIV therapy being administered in between. Any particular combination would be covered by the cycle schedule as long as it is determined that the appropriate schedule involves administration on a certain day.

A pharmaceutical compositions used in the foregoing methods preferably are sterile and contain an effective amount of a therapeutic compound that will induce apoptosis in HIV-infected reservoir cells that produces the desired response (causes an overall reduction of viral load) in a unit of weight or volume suitable for administration to a patient.

The doses of a composition administered to a subject to induce apoptosis in HIV-infected reservoir cells can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

Various modes of administration will be known to one of ordinary skill in the art which effectively deliver a composition to induce apoptosis in HIV-infected reservoir cells. Methods for administering such a composition, or other pharmaceutical compound of the invention may be intravenous, oral, intracavity, intrathecal, intrasynovial, buccal, sublingual, intranasal, transdermal, intravitreal, subcutaneous, intramuscular and intradermal administration. The invention is not limited by the particular modes of administration disclosed herein. Standard references in the art (e.g., Remington's Pharmaceutical Sciences, 18th edition, 1990) provide modes of administration and formulations for delivery of various pharmaceutical preparations and formulations in pharmaceutical carriers. Other protocols which are useful for the administration of therapeutic compound of the invention will be known to one of ordinary skill in the art, in which the dose amount, schedule of administration, sites of administration, mode of administration (e.g., intra-organ) and the like vary from those presented herein.

Administration of a composition comprising a TRAIL receptor activator to induce apoptosis in infected reservoir cells to mammals other than humans, e.g., for testing purposes or veterinary therapeutic purposes, is carried out under substantially the same conditions as described above. It will be understood by one of ordinary skill in the art that this invention is applicable to both human and animal conditions. Thus this invention is intended to be used in husbandry and veterinary medicine as well as in human therapeutics.

The invention is not limited by the particular modes of administration disclosed herein. Standard references in the art (e.g., Remington's Pharmaceutical Sciences, 18th edition, 1990) provide modes of administration and formulations for delivery of various pharmaceutical preparations and formulations in pharmaceutical carriers.

A molecule of the invention that is useful in the treatment of HIV infection may conjugated to or in association with a delivery vehicle such as a nanocarrier. Examples of nanocarriers include, but are not limited to, liposomes, immunolipososomes, microparticles, emulsions, etc. These and other suitable delivery vehicles and methods of their use are known to those of ordinary skill in the art.

When administered, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The methods provided herein include compositions comprising a TRAIL receptor activator, optionally formulated with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with additional HIV or anti-viral drug formulations in a manner such that there is no interaction which would substantially impair the desired pharmaceutical efficacy.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, i.e. EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189. Other polymers that could be used are poly-1,3-dioxolane and poly-1,3,6-tioxocane. Preferred for pharmaceutical usage, as indicated above, are polyethylene glycol moieties.

For the component the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the active agent or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e. powder; for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the active agent may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, a-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethonium chloride. The list of potential non-ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the active agent either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Also contemplated herein is pulmonary delivery of the active agents. The active agent is delivered to the lungs of a mammal while inhaling and traverses across the lung epithelial lining to the blood stream. Other reports of inhaled molecules include Adjei et al., 1990, Pharmaceutical Research, 7:565-569; Adjei et al., 1990, International Journal of Pharmaceutics, 63:135-144 (leuprolide acetate); Braquet et al., 1989, Journal of Cardiovascular Pharmacology, 13(suppl. 5): 143-146 (endothelin-1); Hubbard et al., 1989, Annals of Internal Medicine, Vol. III, pp. 206-212 (a1-antitrypsin); Smith et al., 1989, J. Clin. Invest. 84:1145-1146 (a-1-proteinase); Oswein et al., 1990, “Aerosolization of Proteins”, Proceedings of Symposium on Respiratory Drug Delivery II, Keystone, Colo., March, (recombinant human growth hormone); Debs et al., 1988, J. Immunol. 140:3482-3488 (interferon-g and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of the compound of the invention. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated.

Formulations suitable for use with a nebulizer, either jet or ultrasonic, will typically comprise the compound of the invention dissolved in water at a concentration of about 0.1 to 25 mg of biologically active compound per mL of solution. The formulation may also include a buffer and a simple sugar. The nebulizer formulation may also contain a surfactant, to reduce or prevent surface induced aggregation of the active agent caused by atomization of the solution in forming the aerosol.

Formulations for use with a metered-dose inhaler device will generally comprise a finely divided powder containing the active agent suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing active agent and may also include a bulking agent, such as lactose, sorbitol, sucrose, or mannitol in amounts which facilitate dispersal of the powder from the device, e.g., 50 to 90% by weight of the formulation. The active agent should most advantageously be prepared in particulate form with an average particle size of less than 10 μm (or microns), most preferably 0.5 to 5 μm, for most effective delivery to the distal lung.

Nasal delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The compositions may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The invention will be more fully understood by reference to the following examples. These examples, however, are merely intended to illustrate the embodiments of the invention and are not to be construed to limit the scope of the invention.

### EXAMPLES

In order to establish a persistent infection, viruses have evolved various strategies to subvert immune clearance processes of the host. We present evidence for a mechanism whereby cytokine induction by the HIV-1 envelope glycoprotein protects infected macrophages from death receptor-mediated killing. We demonstrate that the envelope glycoprotein of HIV-1 induces production of the cytokine, monocyte colony stimulating factor (M-CSF) which in turn restricts the expression of the death receptors TRAIL DR4 and TRAIL DR5 on infected, macrophages rendering them resistant to TRAIL-induced apoptosis. In contrast, macrophages infected with an envelope-minus HIV-1 variant were highly sensitive to killing by TRAIL.

TRAIL receptor expression on infected macrophages was increased when M-CSF was inhibited by blocking antibodies or by RNA interference and exogenous MCSF conferred TRAIL resistance on macrophages infected with an envelope-minus HIV-1 variant. In the presence of an intact viral envelope, infected cell viability and virus production were sustained in the presence of high concentrations of TRAIL. Furthermore, we demonstrate that the suppression of death receptors by the viral envelope counteracts TRAIL on the surface of lymphocytes thereby promoting viral dissemination in trans in macrophage lymphocyte co-cultures. Our results suggest that the HIV-1 envelope glycoprotein, which paradoxically is a major determinant of viral cytopathicity in CD4+ lymphocytes, ensures infected macrophage survival and viral dissemination in the face of host apoptotic clearance processes.

An important part of the hosts strategy to prevent viral replication, dissemination or persistence of infected cells involves induction of apoptosis. As a consequence, viruses have evolved countermeasures that either confer resistance to apoptotic cell death or that kill the cells delivering the apoptotic signal. In the case of adenoviruses, the viral E3 protein causes internalization of death receptors (Fas) from the infected cell surface which allows infected cells to resist Fas-mediated cell death. Several herpes viruses including Kaposi's sarcoma-associated herpes virus (KSHV) encode FLICE-inhibitory proteins (VFLIP) which disrupts the recruitment of caspase 8 and the formation of a death-inducing signaling complex. As part of a counter-attack strategy, human cytomegalovirus (HCMV) up regulates death-inducing ligands on the infected cell thereby triggering apoptosis of HCMV-specific T cells. In terms of HIV-1 strategies for immune evasion, most of the attention has focused on mechanisms that are operative in infected lymphocytes. The HIV-1 Nef gene has been shown to block the function of ASK 1 (the apoptosis-signal-regulating kinase 1) on infected lymphocytes to protect infected lymphocytes from Fas and TNF-receptor-mediated apoptosis. At the same time, HIV-1 Nef induces Fas ligand (FasL) expression on infected cells to kill cytotoxic T cells (CTL) that express Fas on the cell surface. Despite the existence of viral mechanisms to protect infected lymphocytes from the host apoptotic response, lymphocytes are rapidly cleared by the cytopathic effects of virus infection. By comparison, the turnover of infected macrophages, which has been extrapolated from decay characteristics of plasma virions during combination antiretroviral therapy and from reported estimates of macrophage life span in the tissues, is much slower (half-life of 2-4 weeks). However, a greater half-life is suggested by studies with highly pathogenic SHIV variants which demonstrate that tissue macrophages can sustain viremia for much greater intervals. Whether there are viral mechanisms that preserve the function of infected macrophages either by counteracting viral cytopathic effects, or host immune clearance processes, is currently unknown. Some macrophage effector functions are modulated by HIV-1 infection and cytokine release is one of the most striking examples. For example, HIV-1 induces the production of the chemokines MIP-1 α and MIP-1β by infected macrophages. This is mediated by the viral Nef protein and has been proposed to promote recruitment of substrate lymphocytes to sites of infection in order to facilitate viral dissemination9. In addition, monocyte colony stimulating factor (M-CSF) is induced upon HIV-1 infection of macrophages in a manner that is Nef-independent. However, the mechanism by which HIV regulates M-CSF production and the role played by M-CSF in viral replication is not well understood.

Thus the work presented herein presents evidence that HIV-1 envelope subverts the host apoptotic response extending macrophage longevity and permitting efficient dissemination of virus to CD4+ T cells.

The envelope glycoproteins of primate lentiviruses harbor regions that mediate the interaction with receptor/core receptor proteins on the surface of susceptible cells as well as determinants which promote fusion between viral and cellular membranes during virus entry (Ray and Doms, 2006). However, some studies suggested activities for human lentiviral envelope proteins beyond their role in virus entry. In particular, signaling through receptor and core receptor molecules following envelope binding may alter target cell function to increases its permissively to virus infection (Cicala et al., 1999; Cicala et al., 2002). In addition, HIV-1 envelope, like other retroviral envelope proteins (Kaplan et al., 2000), contains domains that interact internally with signaling proteins (Martoglio et al., 1997; Micoli et al., 2000) within cells to promote profound changes in cell biology, such as the induction of pro-inflammatory cytokines from CNS cells (Koka et al. 1995). Thus envelope glycoproteins initiate signaling pathways to effect host cell biology and these likely impact viral pathogenesis (Koka et al. 1995; Cicala et al., 2002).

An important modification of host cell function, particularly during chronic viral infections, is the prevention of apoptosis by the infected cell (Barber et al., 2001). As a consequence, viruses have evolved measures that either confer resistance to apoptotic cell death or kill the cells delivering the apoptotic signal. For example, Adenovirus encodes the RID proteins that promote the internalization of death receptors for Fas, TRAIL and TNFR1 to enable infected cells to withstand these apoptotic stimuli (Gooding et al., 1988; Tollefson et al., 1998; Tollefson et al., 2001). As part of a counter-attack strategy, human cytomegalovirus (HCMV) up regulates death-inducing ligands on the infected cell thereby triggering apoptosis of HCMV-specific T cells (Raftery et al., 2001). In HIV-1, most attention has focused on strategies for immune evasion that are operative in infected lymphocytes. The HIV-1 Nef gene has been shown to block the function of ASK 1 (the apoptosis-signal-regulating kinase 1) on infected lymphocytes to protect these cells from Fas and TNF-receptor-mediated apoptosis (Geleziunas et al., 2001). At the same time, HIV-1 Nef induces Fas ligand expression on infected cells to kill cytotoxic T cells that express Fas on the cell surface (Fackler and Baur, 2002). Despite the existence of viral mechanisms to protect infected lymphocytes from the host apoptotic response, lymphocytes are rapidly cleared by the cytopathic effects of virus infection (Perelson et al., 1996). By comparison, the turnover of infected macrophages, which has been extrapolated from decay characteristics of plasma virions during highly active antiretroviral therapy (HAART) and from reported estimates of macrophage life span in the tissues (Perelson et al., 1996), is far slower (half-life of 2-4 weeks). However, greater half-life is suggested by studies with highly pathogenic SHIV variants which demonstrate that tissue macrophages can sustain viremia for prolonged intervals (Igarashi et al., 2001).

HIV-1 infected macrophages release the pro-survival cytokine macrophage colony stimulating factor, M-CSF in addition to the β-chemokines, MIP-1α and MIP-1β (Gruber et. al., 1995; Swingler et al, 1999). Whilst chemokines result in the attraction of T lymphocytes to HIV-1 infected macrophages and promote the transfer of virus to CD4+ T cells (Swingler et al., 1999; Swingler et al., 2003), the role of M-CSF is unclear. However it is likely that chemoattraction also recruits effector cells capable of a cytotoxic response to HIV-1 infected macrophages. We investigated whether survival signals from M-CSF or those engendered by the envelope glycoprotein increase lymphocyte permissively and exert positive effects on both the dissemination of HIV-1 to CD4+ T lymphocytes and viability of the infected macrophages.

Evidence presented herein shows that the HIV-1 glycoprotein elicits a potent prosurvivalresponse in infected macrophages that specifically blocks their vulnerability to destruction by the apoptotic ligand TRAIL. As a result, HIV-1 infected macrophages are able to sustain high level virus replication in the presence of TRAIL on cytotoxic lymphocytes without which, macrophage survival and dissemination of virus to substrate T lymphocytes is significantly impacted. Furthermore, the anti-leukemia drug, Imatinib, disabled HIV-1 mediated resistance to TRAIL, and permitted the induction of apoptosis in infected macrophages. This suggested a novel role for this drug to promote apoptosis of HIV-1 infected macrophages and potentially eliminate viral reservoirs in macrophages that survive in the face of HAART.

### Results

### HIV-1 Envelope Regulates M-CSF Production and Controls Death Receptor Expression

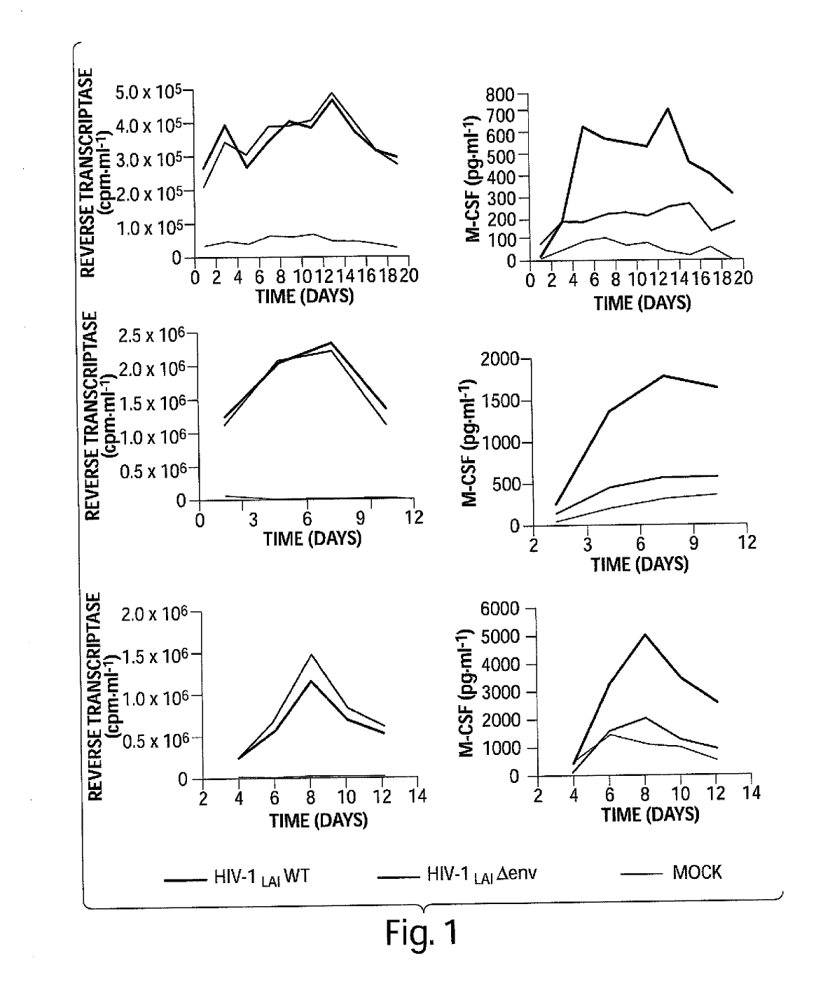

In order to characterize the biological role of M-CSF in HIV-1 replication, we initially identified the viral gene product responsible for its induction. Viruses containing inactivating mutations in structural and accessory genes in an X4-tropic HIV-1 LAI clone, were pseudotyped with vesicular stomatitis virus (VSV) envelope to allow single cycle infection of macrophages. Levels of virus production (extracellular reverse transcriptase activity) and M-CSF release was followed at different intervals after infection. While Vpu, Vif, Nef, and Vpr were relatively dispensable for the induction of M-CSF the release of M-CSF by macrophages infected with an HIV-1 envelope-minus variant (HIV-1 LAI Δenv) was impaired relative to macrophages infected with a wild-type virus (FIG. 1). M-CSF induction by wild-type infected macrophages appeared to be post-transcriptional as no change in M-CSF mRNA levels were detected. As used herein, viral mutants (variants) lacking functional envelope is referred to as “envelope-minus,” “Δenvelope” or “Δenv,” all of which are used interchangeably.

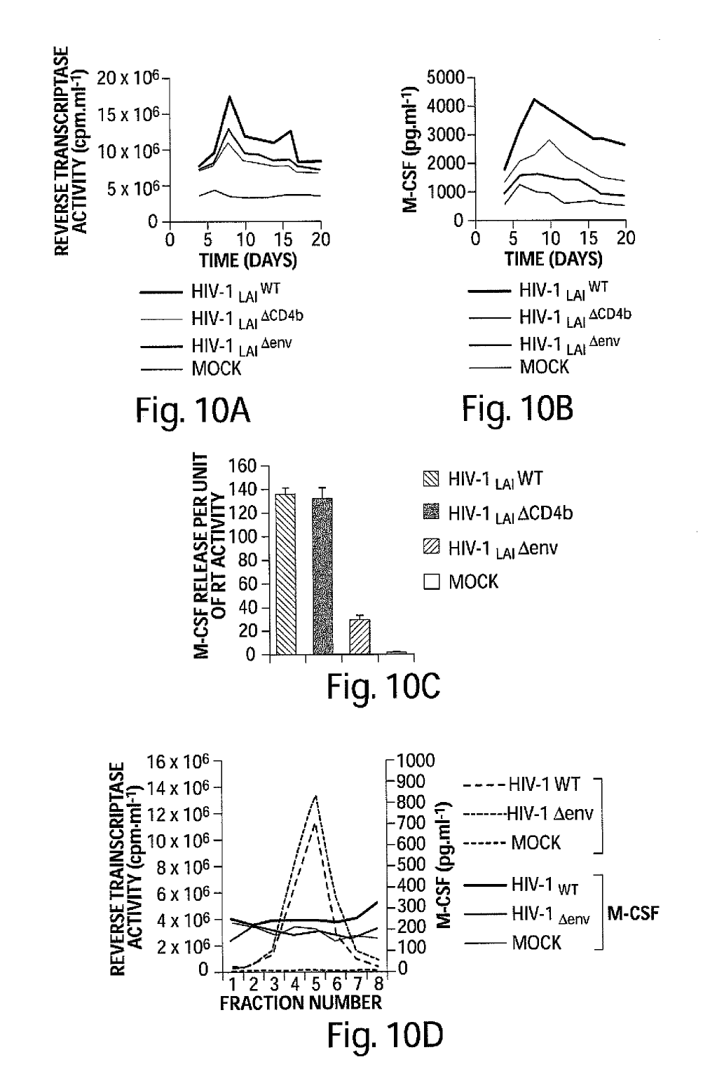

HIV-1 envelope has been shown to induce signals through the chemokine receptors (Weissman et al., 1997; Davis et al., 1997) which could influence the expression of cellular genes. However, M-CSF was induced upon infection of macrophages with viruses harboring a CXCR4-tropic envelope (FIG. 1) or an R5 tropic envelope (Swingler et al., 1999). To conclude that the envelope dependent M-CSF production was independent of CD4 and co-receptor usage, macrophages were infected with a pseudotyped HIV-1 variant HIV-1 LAI ΔCD4b that harbors a mutation in the CD4 binding epitope of envelope (Thali et al., 1991). M-CSF production by macrophages infected with the CD4 binding mutant was not significantly lower than that from macrophages infected with the pseudotyped wild-type virus, however, it was substantially greater than by the envelope-minus variants (see FIG. 10). In addition, M-CSF was not induced when macrophages were pulsed with gradient purified R5-tropic HIV-1 ADA virions or with HIV-1 ADA Δenv virions (FIG. 11). Collectively, these results indicate that induction of M-CSF by the viral envelope glycoprotein is independent of receptor or co-receptor interactions and that the viral envelope synthesized de novo within infected macrophages but not virion-associated envelope induces M-CSF production.

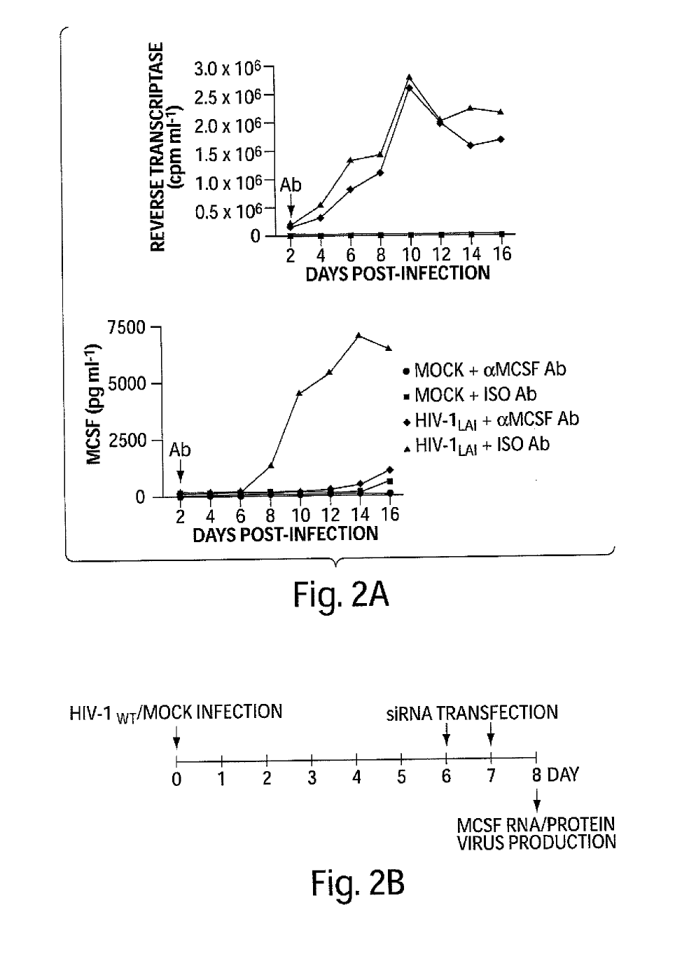

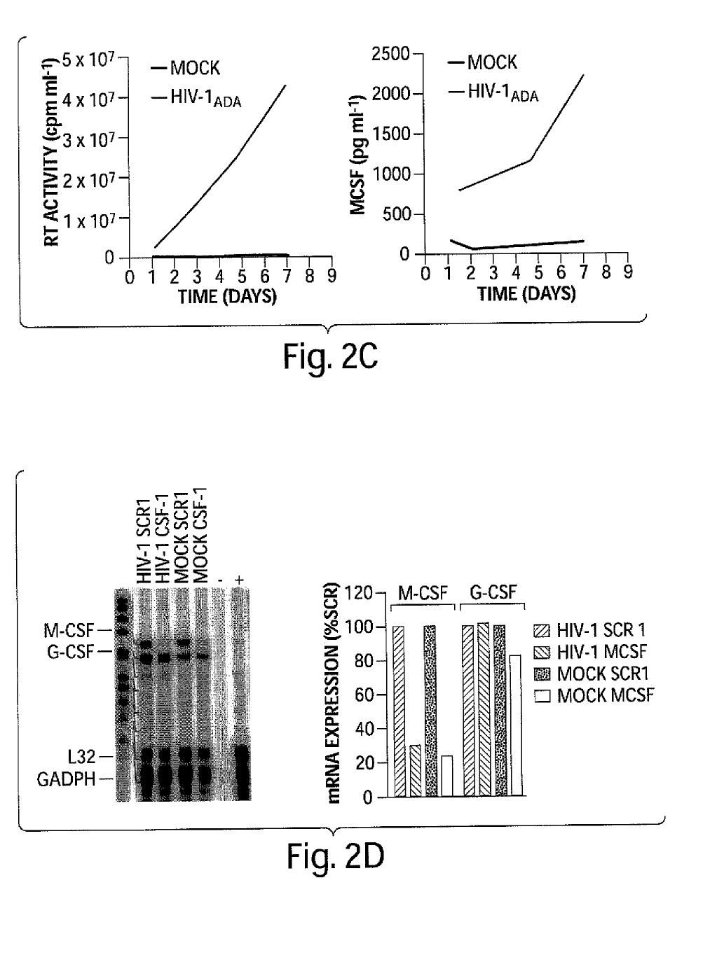

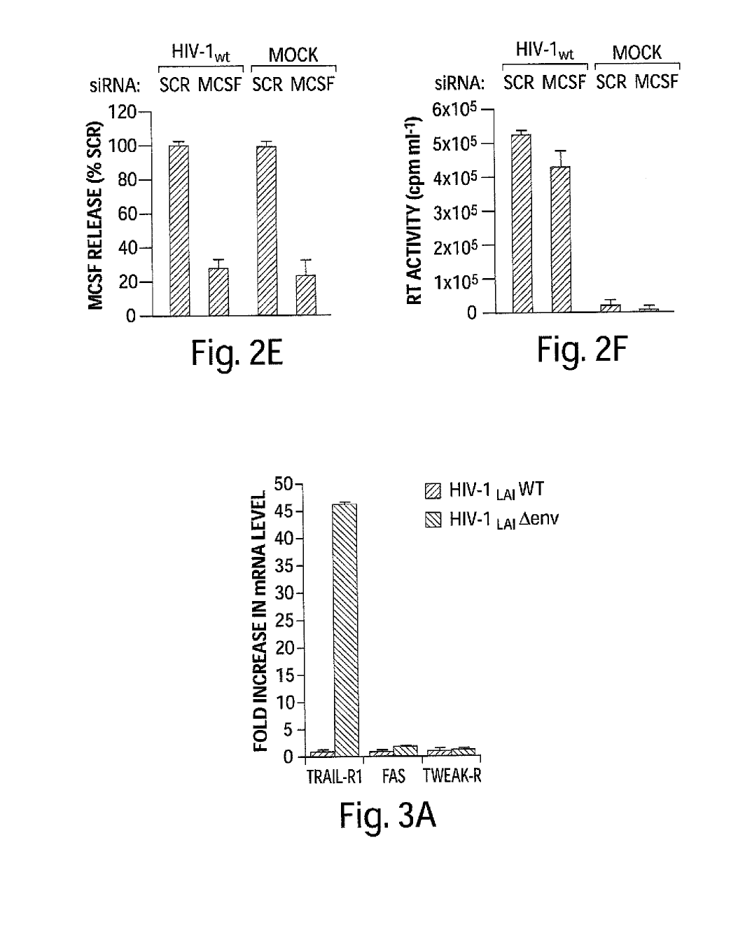

To determine whether M-CSF induction by HIV-1 infected macrophages directly impacted virus production, two independent approaches were used to regulate M-CSF production. Firstly, macrophages were infected with pseudotyped HIV-1 LAI, then M-CSF produced by the infected macrophages was neutralized by addition of an M-CSF-specific antibody. Despite efficient neutralization of M-CSF activity (FIG. 2A, right panel), virus production continued at levels seen in macrophages treated with an isotype antibody (FIG. 2A, left panel). Secondly, M-CSF production was inhibited by RNA interference (FIG. 2B). Macrophages were infected with a wild-type HIV-1 R5 tropic variant (HIV-1 ADA) and monitored for viral replication (FIG. 2C, upper panel) and M-CSF release (FIG. 2C, lower panel). At 6 and 7 days post infection, when replication and M-CSF approached peak levels, macrophages were transfected with M-CSF-specific siRNAs or with a scrambled siRNA. Introduction of M-CSF-specific siRNAs resulted in an ˜80% reduction in M-CSF transcripts relative to G-CSF transcripts in transfected macrophages (FIG. 2D) as well as an ˜80% reduction in M-CSF release (FIG. 2E). Nevertheless, there was no significant effect on virus production under these conditions (FIG. 2F). Therefore, in the context of a single cycle infection or a spreading infection, M-CSF did not appear to influence impact the efficiency of virus production by infected macrophages. These results suggest that contrary to a previous report (Kutza et al, 2000), neutralization of M-CSF did not affect susceptibility of macrophages to HIV-1 infection nor subsequent viral replication.

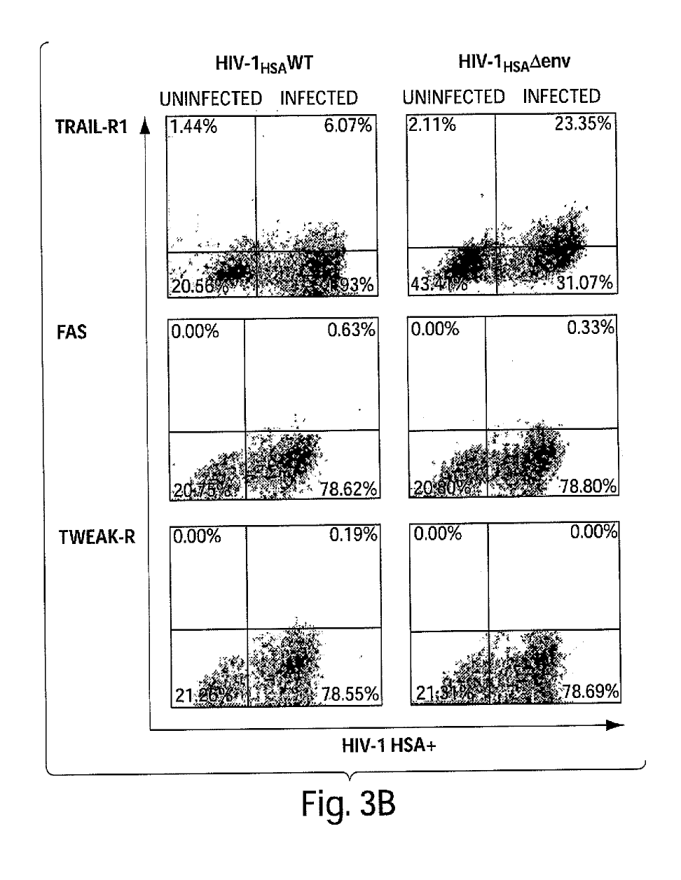

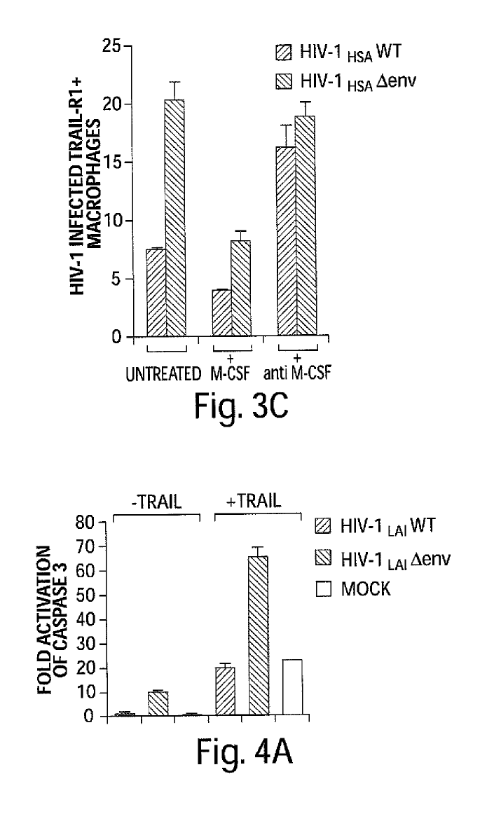

Therefore, macrophages infected with pseudotyped wild-type and envelope minus HIV-1 variants were further examined using pathway specific gene arrays in order to identify cellular genes whose expression was affected by HIV-1 envelope and reveal a possible role for M-CSF release. Cytokines can counteract apoptotic signals (Derouet et al., 2004; Li et al., 2004) and analysis of genes involved in apoptosis revealed that macrophages infected with a envelope minus virus but not wild type expressed a greatly elevated level of death receptor for TRAIL (TRAIL-R1/DR4). RNA for TRAIL-R1 receptor was increased over 45-fold in macrophages infected with Δenvelope relative to wild type virus (FIG. 3A). Similarly, surface expression of TRAIL-R1 was detected on 23.4% of HIV-1 Δenvelope infected macrophages compared to 6.1% on wild type infected macrophages (FIG. 3B). In contrast, expression of other death receptors for Fas ligand and TWEAK were not upregulated (FIGS. 3A & 3B).

Most notably, when M-CSF from macrophages infected with wild type R5 tropic HIV-1 ADA or pseudotyped X4 tropic HIV-1 LAI was neutralized by antibody, surface TRAIL-R1 expression was increased (FIG. 3C). Conversely, exogenous M-CSF down-regulated TRAIL-R1 on macrophages infected with an envelope defective virus (FIG. 3C). TRAIL-R1 expression on macrophages infected with wild type virus could similarly be increased if M-CSF production was inhibited by RNA interference. Messenger RNA and surface expression of the functionally equivalent TRAIL Receptor 2 (TRAIL-R2/DR5) (Zhang and Fang, 2005) was only modestly up-regulated (less than two-fold) on macrophages infected with an envelope minus variant. TRAIL-R2, like R1, was expressed at equivalently low levels on macrophages infected with wild type HIV-1 or uninfected (FIG. 3C). These data confirmed that the viral envelope glycoprotein, through the induction of M-CSF, suppressed expression of these receptors, principally TRAIL-R1, on the surface of HIV-1 infected macrophages.

### Modulation of TRAIL Receptors Prevents Apoptosis and Prolongs Macrophage Longevity

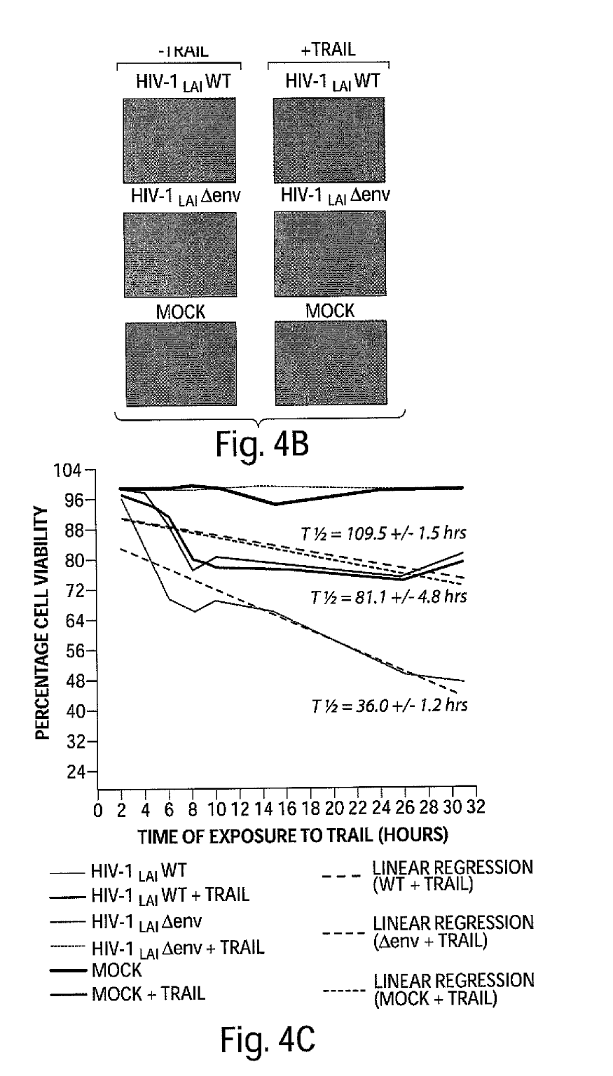

The expression of death receptors for TRAIL correlated with the relative sensitivity of HIV-1 WT and HIV-1 Δenv infected macrophages to TRAIL-mediated apoptosis (FIG. 4A) as evidenced by increased levels of active (cleaved) caspase 3; a central mediator of mediator of TRAIL-induced apoptosis (Zhang and Fang, 2005). Macrophage cultures infected with pseudotyped envelope minus HIV-1 succumbed within 6 hours to visible deleterious effects of biologically active TRAIL, whereas wild type infected cultures, like mock, remained comparatively unaffected (FIG. 4B).

Macrophages are a long-lived reservoir of HIV in vivo and a source of viral rebound (reviewed in Stevenson, 2003). The evasion of apoptosis in other human viral infections contributes to viral persistence and enables the infected cell to survive in the face of a hostile microenvironment (Raftery et al., 2001; Secchiero et al., 2001; Tollefson et al. 1998; Tollefson et al. 2001). Therefore we determined the half-life of HIV-1 infected macrophages in the presence of a concentration of TRAIL capable of inducing apoptosis. Macrophages infected with an envelope minus virus, which are unable to produce significant M-CSF and suppress DR4 expression, exhibited greatly impaired viability in these conditions compared to HIV-1 WT or Mock infected cells. The half-life of HIV-1 Δenv infected macrophages was reduced to around 36 hours compared to 110 and 81 hours for wild type and mock infected cultures respectively (FIG. 4C). Thus resistance to TRAIL can potentially provide a significant advantage to macrophages infected by HIV-1.

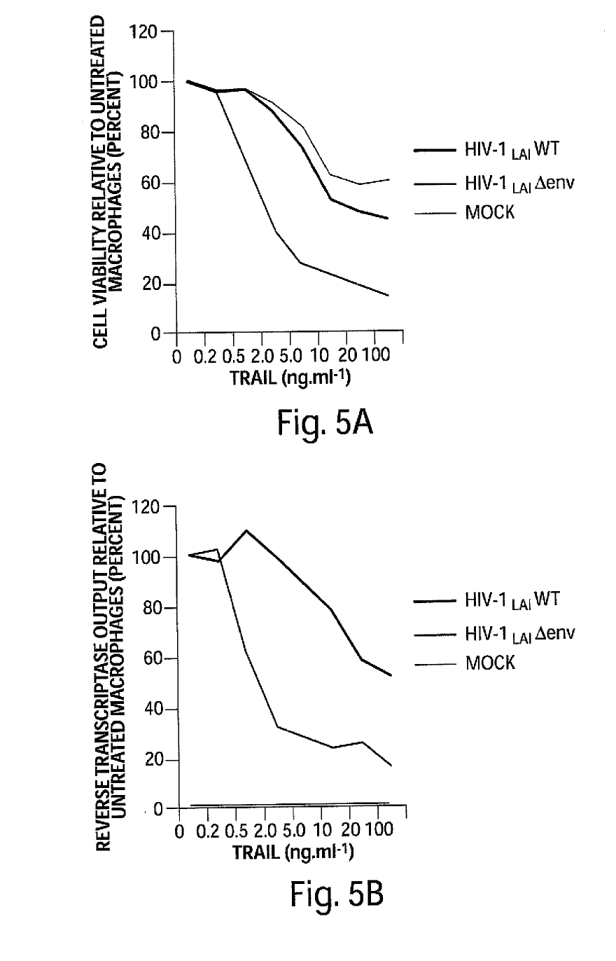

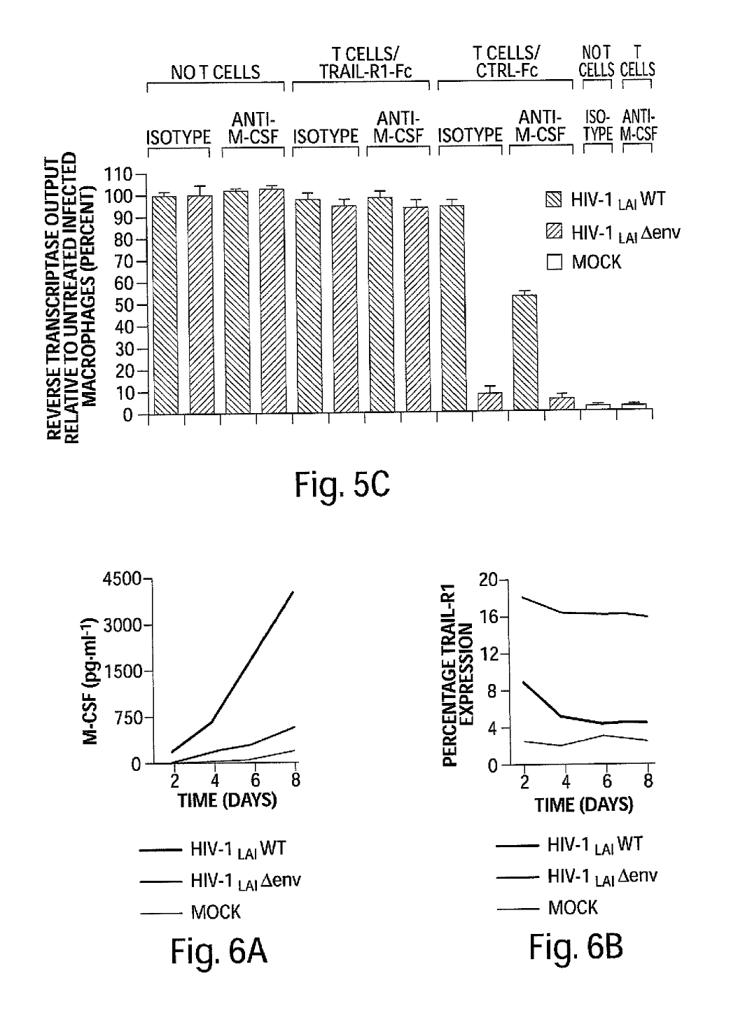

The differential sensitivity of wild type and envelope minus HIV-1 infected macrophages to TRAIL-mediated cell killing correlated with the concentration of TRAIL required to impact cell viability and virus production. In macrophages infected with pseudotyped HIV-1 LAI Δenv, a TRAIL concentration of 3 ng·ml-1 effected a ˜50% reduction in cell viability and virus output (FIGS. 5A & 5B). In contrast, in excess of 30 ng·ml-1 and 50 ng·ml-1 of TRAIL respectively were required for a similar reduction viability and virus output from macrophages infected with pseudotyped wild type HIV-1 LAI (FIGS. 5A & 5B). In an environment were effector T lymphocytes could provide a restriction on virus output through TRAIL, activated CD8+ T cells (˜16% TRAIL+) were co-cultured with infected macrophages (FIG. 5C). Isolated CD8+ T cells were used to avoid any T cell contribution to viral replication. The virus output of HIV-1 Δenv infected macrophages was reduced by ˜90% and was dependent on TRAIL present on T lymphocytes. Viral production by wild type infected macrophages was largely unaffected, although it was reduced to ˜40% when M-CSF was neutralized by antibody. As inactivation of macrophage-derived M-CSF did not equally inhibit virus production from wild type infected cells compared to those infected with a Δenv variant virus, it suggested that envelope glycoprotein-mediated resistance to apoptosis by TRAIL through mechanisms additional to M-CSF induction.

### HIV-1 Envelope Induces Host Anti-Apoptotic Proteins

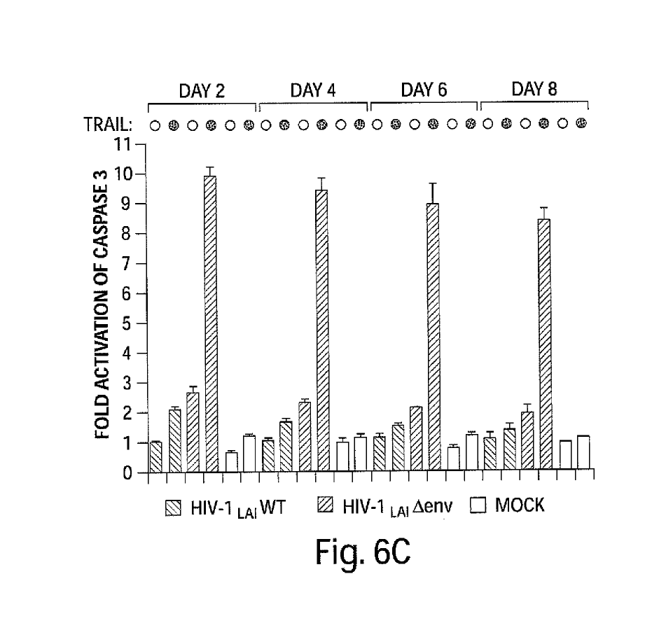

To further investigate the relationship between M-CSF release, TRAIL-R1 expression and TRAIL-dependent apoptosis, these parameters were examined over the course of viral replication in HIV-1 infected macrophages (FIG. 6). Macrophages infected with a pseudotyped envelope minus HIV-1 replicated virus equivalently to the wild type variant but did not significantly induce M-CSF and maintained a high level of TRAIL-R1 expression (FIGS. 6A & 6B). HIV-1 Δenv infected macrophages were acutely sensitive to TRAIL-mediated apoptosis at an early time point (Day 2) and through all later time points, as shown by an 8 to 10 fold increase in active caspase 3 (FIG. 6C). However, when M-CSF production by HIV-1 WT infected macrophages was low early in infection, these cells did not exhibit the sensitivity to TRAIL of HIV-1 Δenv infected macrophages. In HIV-1 WT infected macrophages, TRAIL-induced active caspase 3 was elevated 3 fold at Day 2 and progressively decreased over time (FIG. 6C), with increasing release of M-CSF (FIG. 6A) and falling TRAIL-R1 expression (FIG. 6B). The ability of HIV-1 WT infected macrophages to maintain substantial resistance to TRAIL in the relative absence of M-CSF production, despite higher TRAIL-R1 expression, further indicated that the envelope glycoprotein prevented apoptosis by TRAIL through broader processes.

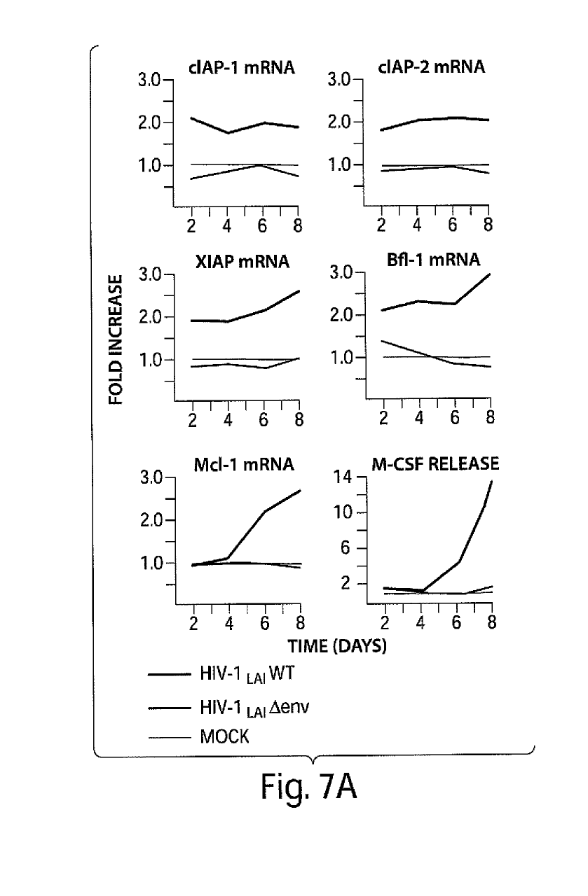

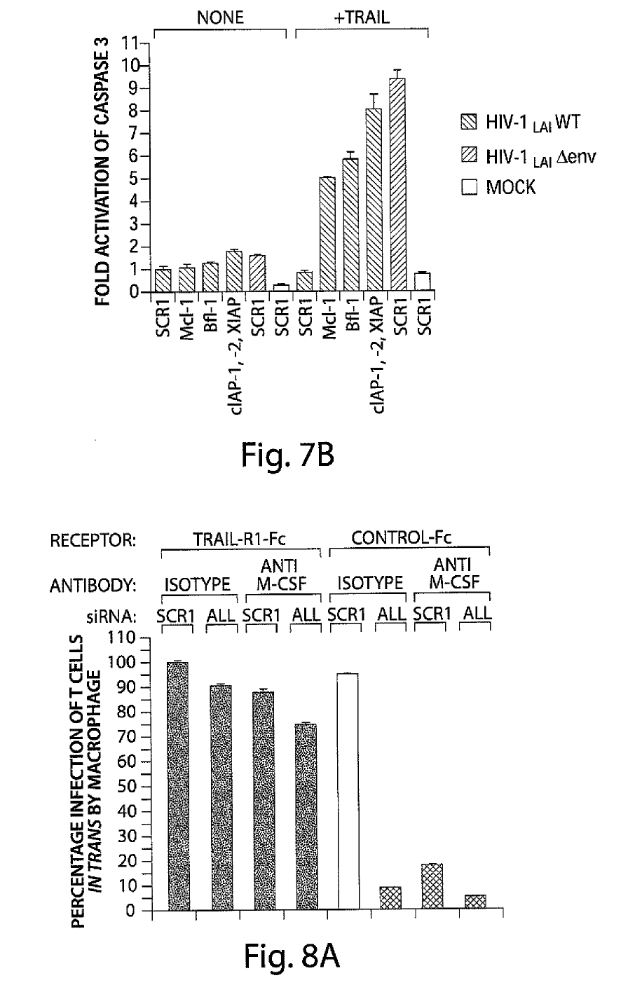

In HIV-1 infected macrophages, the expression of cellular genes that participate in the regulation of the TRAIL death pathway were examined further by targeted gene arrays. We observed that mRNAs encoding five anti-apoptotic genes, in addition to M-CSF, were significantly upregulated only in macrophages infected with a virus containing an intact envelope gene (FIG. 7A). The mRNA for three BIRC family proteins, cIAP-1, cIAP-2 and XIAP which antagonize caspase activation (Salvesen and Duckett, 2002; Zhang and Fang, 2005) were increased, along with mRNA for two antiapoptotic Bcl family proteins, Bfl-1 and Mcl-1, which inhibit mitochondrial dependent apoptosis (Zhang and Fang, 2005; Han et al., 2006). Mcl-1 expression paralleled M-CSF release and was the only member induced by M-CSF as demonstrated when exogenous M-CSF was added to macrophages infected with envelope minus HIV-1. These data confirmed that envelope transmitted both M-CSF independent and M-CSF dependent signals. Inhibition of Mcl-1, Bfl-1 or the IAP genes by RNA interference caused a significant reduction in the resistance of wild type infected macrophages to TRAIL (FIG. 7B). Individually, Mcl-1 and Bfl-1 allowed TRAIL to induce apoptosis in HIV-1WT infected macrophages, assessed by active caspase 3, to ˜50% and ˜60% respectively of HIV-1 Δenv infected cells (FIG. 7B). Likewise, inhibition of the IAP genes raised TRAIL-dependent apoptosis of wild type infected macrophages to ˜75% of the envelope minus infected cells. Thus, the regulation of TRAIL-mediated apoptosis by HIV-1 envelope occurred at multiple levels and in conjunction with M-CSF and the modulation of surface TRAIL-R1 expression (Summarized in FIG. 11).

### Viral Dissemination, Macrophage Survival and the Reversal of TRAIL Resistance by Imatinib

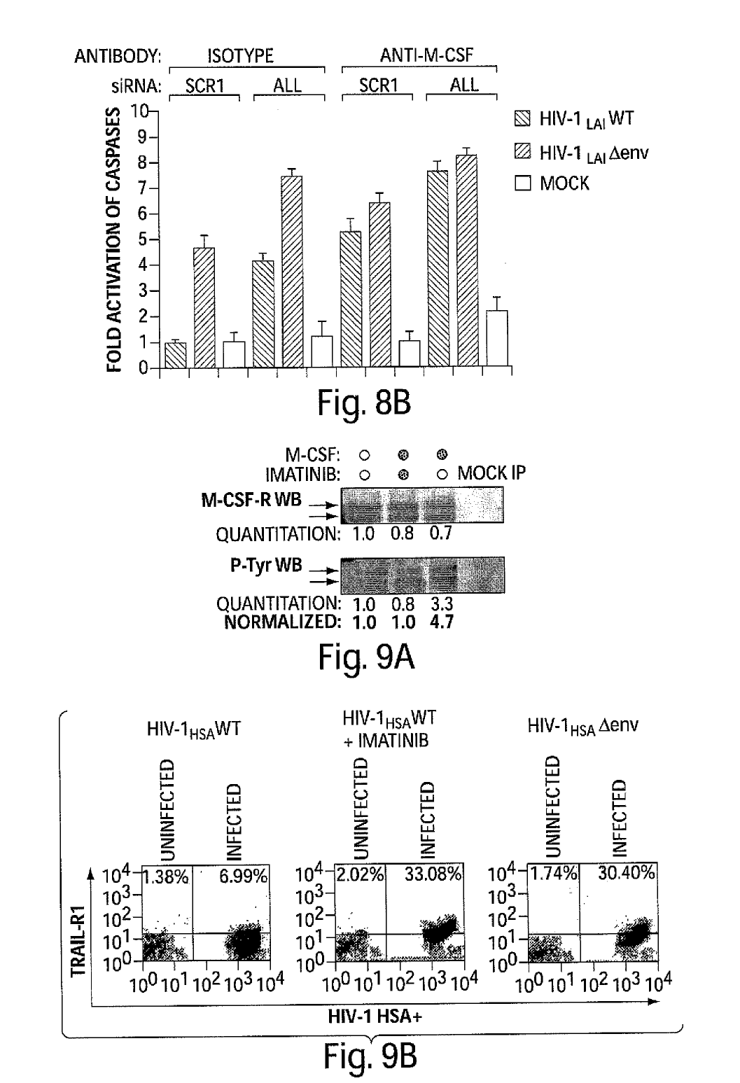

Infected macrophages efficiently transfer HIV to substrate lymphocytes (Carr et al. 1999; Swingler et al. 1999; Swingler et al. 2003) and macrophage reservoirs are a likely source of rebounding virus when HAART fails (Sharova et al 2005; Stevenson, 2003). Surprisingly, it was the ability of macrophages infected with wild type HIV-1 to resist a hostile environment provided by TRAIL that was responsible for the vast majority of de novo infection of activated T lymphocytes in trans (FIG. 8A). Removal of the defenses provided by envelope against TRAIL, through neutralization of M-CSF and RNA interference to the anti-apoptotic genes, Mcl-1, Bfl-1, cIAP-1, cIAP-2 and XIAP, caused ˜95% reduction in viral dissemination from infected macrophages to activated T lymphocytes (˜21% TRAIL+) during co-culture (FIG. 8A).

Macrophages infected with an envelope minus virus, which is unable to initiate T cell infection, served as a control for contaminating macrophages in the isolated lymphocytes analyzed. Pre-incubation of T cells with soluble recombinant TRAIL-R1 demonstrated that the inhibition of T cell infection in trans was dependent on TRAIL expressed on the surface of activated T cells, and was greatest when both M-CSF dependent and M-CSF dependent mechanisms in macrophages were disabled (FIG. 8A). Infected macrophages similarly co-cultured with activated T lymphocytes pre-incubated with soluble recombinant Fas or TWEAK receptor did not augment the infection of T cells in trans indicating that TRAIL was the major determinant. TRAIL+stimulated T lymphocytes acted by inducing HIV-1 infected macrophages to undergo apoptosis when M-CSF was neutralized or when envelope-dependent host anti-apoptotic genes were inhibited by RNA interference (FIG. 8B). Apoptosis could be detected by the generation of activate caspases in the HIV-1 infected CD14+ monocyte/macrophage populations after co-culture with T cells. TRAIL+activated T cells increased apoptosis in wild type infected macrophages approximately 4 to 5 fold following either neutralizing antibody to M-CSF or siRNAs to the anti-apoptotic genes involved (FIG. 8B). The combination of antibody and RNAi treatments was sufficient to raise macrophage apoptosis to a level equivalent to HIV-1 Δenv infected macrophages (FIG. 8B). Under the more physiological exposure to TRAIL on lymphocytes, it was evident that both envelope induced M-CSF and envelope-dependent host anti-apoptotic gene expression made broadly equivalent contributions to the TRAIL resistance of HIV-1 infected macrophages (FIG. 8B). The induction of macrophage apoptosis by activated T lymphocytes was mediated by TRAIL since pre-incubation with soluble recombinant TRAIL-R1 prior to co-culture could prevent caspase activation.

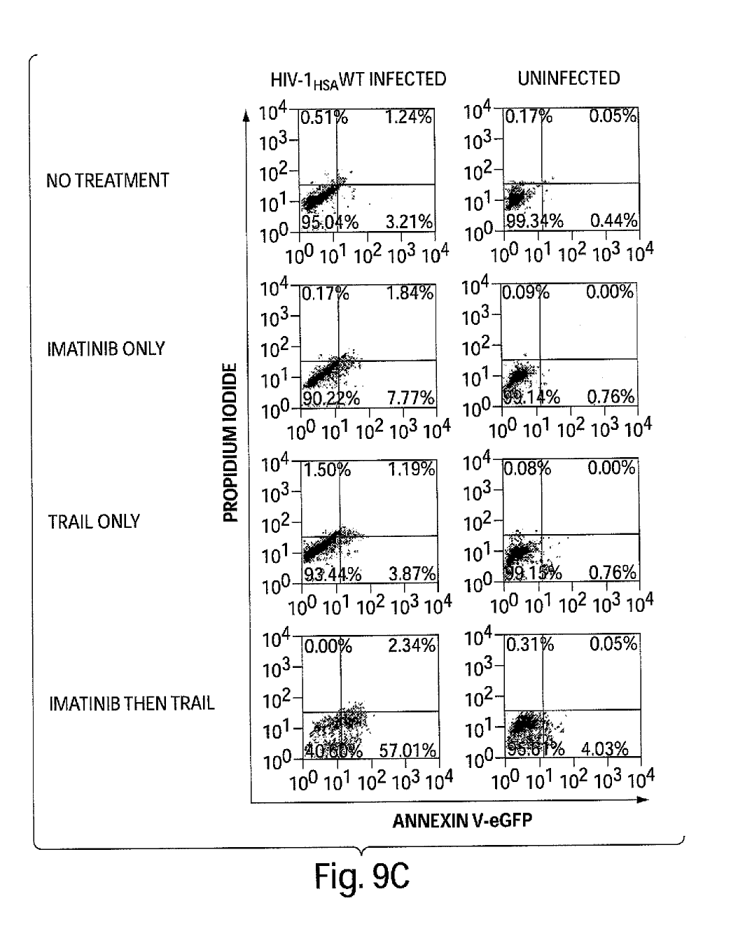

The anti-leukemia drug, Imatinib, inhibits the tyrosine kinase activity of the bcr-abl oncogene and also shows considerable selectivity and efficacy towards the intrinsic kinase activity of the M-CSF receptor (Taylor et al., 2006). Since inhibition of M-CSF by neutralizing antibody was able to induce TRAIL-R1 (FIG. 3C) and cause TRAIL-dependent apoptosis of normally resistant HIV-1 infected macrophages (FIG. 8B), Imatinib was examined as a potential anti-HIV therapeutic agent. The M-CSF receptor undergoes auto-phosphorylation after ligand binding (Taylor et al., 2006) and in human primary macrophages, Imatinib at concentrations obtainable in patients, efficiently inhibited receptor phosphorylation (FIG. 9A). This suggested that Imatinib could block signaling downstream of the M-CSF receptor in cells infected by HIV. Treatment of HIV-1 WT infected macrophages with Imatinib was extremely effective in inducting the surface expression of TRAIL-R1 (FIG. 9B). TRAIL-R1 was induced on 33.1% of HIV-1 WT infected macrophages, compared to 30.4% observed on untreated macrophages infected with an envelope minus HIV-1. This result indicated that Imatinib abolished the ability of envelope, via M-CSF, to down-regulate TRAIL-R1 expression. Imatinib did not promote TRAIL-R1 expression on uninfected macrophages and more importantly, those uninfected macrophages present in the cultures of infected cells (FIG. 9B).

The ability of Imatinib to render HIV-1 WT infected macrophages susceptible to TRAIL was examined by Annexin V and propidium iodide staining so that both apoptotic and late apoptotic/necrotic cell death could be assessed (FIG. 9C). Without Imatinib treatment, infected macrophages underwent a low level of apoptosis (3.2%) which, as expected, was not enhanced by TRAIL exposure (3.9%) (FIG. 9C). When macrophages were exposed to TRAIL following Imatinib treatment, apoptosis was induced in 57% of HIV-1WT infected macrophages and necrosis in 2.3%. In comparison, the uninfected macrophages present in the same culture demonstrated a very small (4.0%) proportion of apoptotic cells and negligible necrosis (FIG. 9C). The 57% induction of apoptosis seen in wild type infected macrophages after Imatinib and TRAIL was equivalent to the maximum achieved using the direct chemical inducers of apoptosis, Staurosporine and Apoptosis Activator I. Therefore Imatinib is effective in substantially degrading the resistance of HIV-1 infected macrophages to TRAIL and acts specifically to render only the infected cells highly susceptible to apoptosis. This suggests that Imatinib, in concert with the innate immune system or exogenous TRAIL, may be a viable therapeutic agent to target HIV-1 infected macrophages in vivo.

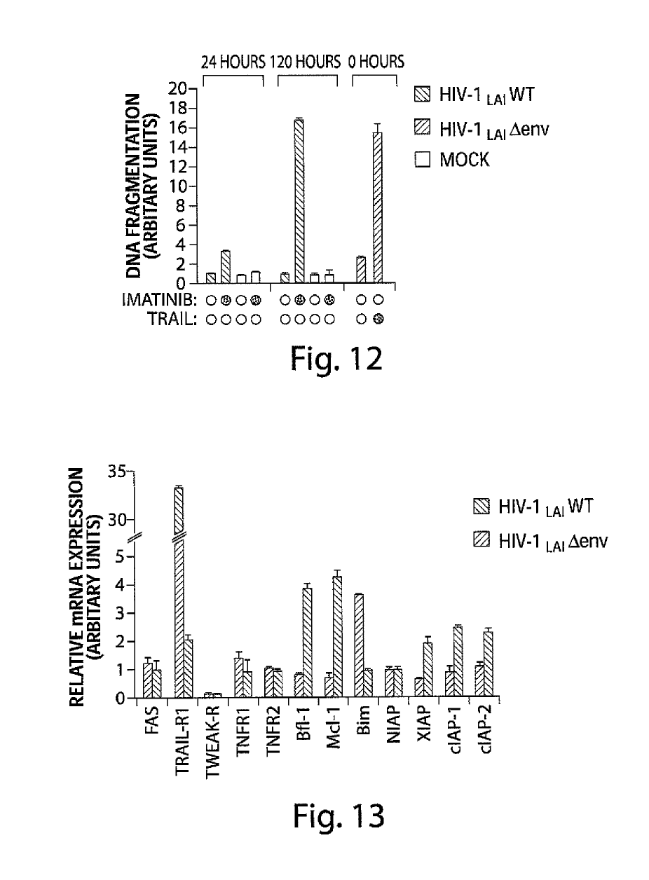

Furthermore, as shown in FIG. 12, the data suggest that Imatinib alone is sufficient to counteract the survival signals that keep HIV-1-infected macrophages alive. In these experiments, five days after infection with HIV-1 LAI wild type or Δenvelope viruses, macrophages were incubated with Imatinib for 24 or 120 hours, and levels of apoptosis were determined by ELISA for histone-associated cellular DNA fragmentation in macrophage lysates (Cell Death ELISA; Error bard, SD) (FIG. 12). Results presented in FIG. 12 show that long-term exposure to Imatinib results in apoptosis of wild-type-infected macrophages even in the absence of exogenously added TRAIL ligand.

### The Mode of Action

To examine whether envelope synthesis inside the infected cell accounted for the observed induction of M-CSF; alternatively, whether virion envelope binding to CD4 or co-receptor was responsible for the effect, M-CSF induction by a CD4-binding mutant of HIV-1 was assayed. FIG. 10 shows the results of virus production (FIG. 10A) and M-CSF release (FIG. 10B). Data were collected at various intervals following infection with pseudotyped HIV-1 variants containing intact or deleted envelope genes or an HIV-1 variant (HIV-1 LAI ACD4b) lacking a functional CD4 receptor binding motif in envelope. Cumulative M-CSF release (shown in FIG. 10C) during the course of viral replication was determined by normalizing the amount of M-CSF to Reverse Transcriptase output and illustrated by bar chart (error bars, SD). FIG. 10D shows effect of cell free HIV-1 virions on M-CSF production by macrophages. R5-tropic HIV-1ADAWT and VSV-pseudotyped HIV-1 ADA Δenv virions were purified on a continuous 15-60% sucrose gradients. Individual gradient fractions were dialyzed, analyzed for reverse transcriptase activity and added to macrophage cultures for 1 hour and M-CSF production determined after 16 hours later. Gradient fractions of mock-infected macrophage supernatants were used as controls.

Collectively, these data demonstrate that M-CSF was induced by de novo synthesis of the viral envelope protein within the infected cell, not by virus particles binding macrophage CD4 or co-receptor. Consistent with this notion, results from a similar set of experiments also showed that the induction of anti-apoptotic gene expression by envelope was due to de novo synthesis and not by virus particles binding macrophage CD4 or co-receptor.

### Imatinib Induces Expression of Apoptosis-Associated Proteins

It has been shown that envelope induces over-expression of anti-apoptotic genes, Mcl-1, Bfl-1, cIAP-1, cIAP-2 and XIAP, that function in preventing mitochondrial and caspase-mediated apoptosis by TRAIL (Salvesen and Duckett, 2002; Zhang & Fang, 2005; Han et al., 2006). Work presented herein further provides evidence that Imatinib treatment of HIV-1-infected macrophages essentially reverts the balance of the expression of bcl family of proteins. To demonstrate this, macrophages infected with pseudotyped HIV-1 LAI WT or HIV-1_{LA1} Δenvelope viruses were incubated with 2 μM of Imatinib for 16 hours, and subsequently the levels of pro- and anti-apoptotic proteins (Bcl-2, Mcl-1, BclX_{L}, BimL, Bmf nad cFLIP) in the TRAIL signaling pathway was determined by Western blotting. As shown in FIG. 11, Imatinib treatment appears to cause a shift in the expression of the proteins in favor of cellular apoptosis.

Consistent with the notion, a number of genes implicated to play a role in cellular apoptosis are affected by HIV-1 envelope. As shown in FIG. 13, which provides relative mRNA levels of apoptosis-associated genes, HIV-1 envelope regulates host genes that are involved in the control of apoptosis. Messenger RNA levels were compared in cDNA gene arrays between macrophages infected with pseudotyped X4 wld type HIV-1 and a Δenvelope variant five days post infection. Gene expression was considered significantly different when the variation was >1.7 units (for detailed analytical methods, see Butte et al., 2001).

### Discussion

In the work described herein, evidence is presented for a novel mechanism that permits the persistence of HIV-1 infected macrophages in the face of unfavorable environmental signals and a potential chemo-therapeutic agent that is able to counter-act this viral defense. The dissemination of HIV from infected macrophages to neighboring T lymphocytes requires intimate contact which leaves infected macrophages susceptible to death signals delivered by TRAIL on lymphocytes. TRAIL cytotoxicity is mediated by CD8+ and CD4+ T lymphocytes and NK cells (Kayagaki et al., 1999; Mirandola. et al., 2004). In vivo macrophages can undergo apoptosis upon activating CD4+ T cells and the induction of macrophage apoptosis by TRAIL on CD4+ T cells has been proposed as part of macrophage homeostasis during antigen presentation (Kaplan et al., 2000). The regulation of activated macrophages by TRAIL also serves to limit immune responses as well as target macrophages infected with intracellular organisms for elimination (Richardson et al., 1993).

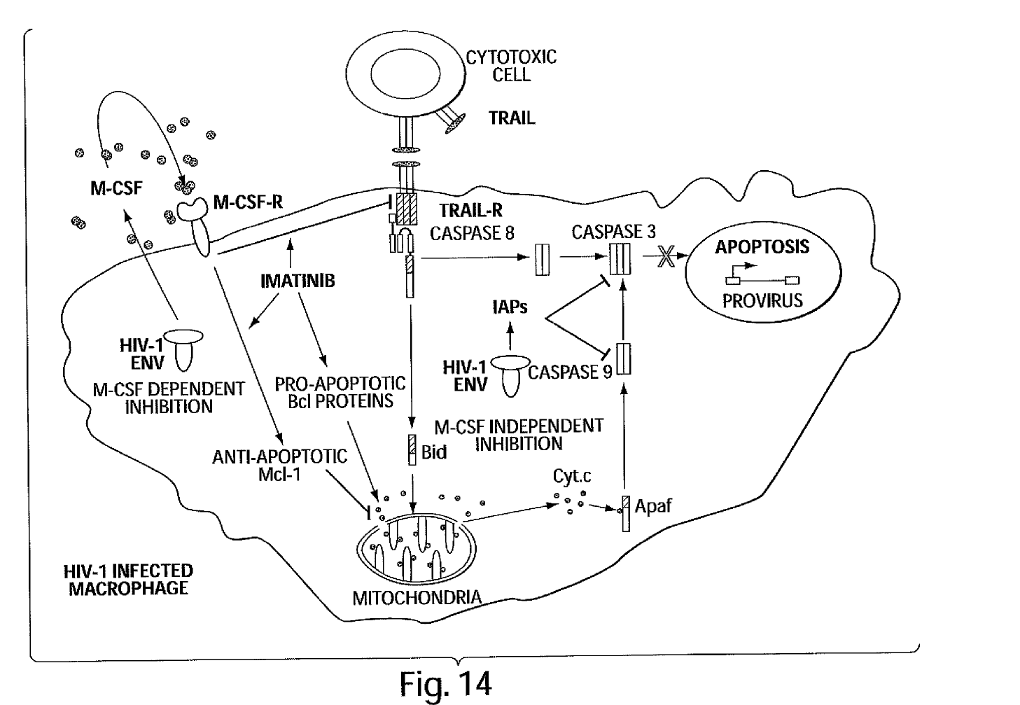

As illustrated in FIG. 14, work described herein supports the notion that Imatinib favors pro-apoptotic gene expression that suggests it is the activation of the mitochondrial pathway that mediates sensitivity to TRAIL after Imatinib treatment.

Thus, the results presented herein indicate that HIV-1 subverts innate immunity to favor macrophage survival. While it has been suggested that TRAIL agonists may be exploited to purge macrophage reservoirs of HIV-1 in infected individuals (Lum et al., 2001), these findings argue that HIV-1 infected macrophages will be resistant to such an approach without the administration of the therapeutic methods described herein. However, the intrinsic resistance of HIV-1 infected macrophages to TRAIL can be disabled by the drug Imatinib. Like the neutralization of macrophage M-CSF by antibody, this anti-cancer drug blocks M-CSF signaling and restores TRAIL-R1 expression and, in addition, can up-regulate pro-apoptotic Bcl family members (Kuroda et al., 2006), one of which antagonizes the anti-apoptotic Mcl-1 gene (Han et al., 2006) that is also regulated by HIV-1 envelope. Based on these findings presented herein, exogenous TRAIL or activated T lymphocytes, used as a source of antigen non-specific TRAIL+immune effector cells, can provide the apoptotic stimuli capable of eliminating infected macrophages and offer a potential new avenue of therapy. Imatinib has been demonstrated to augment the susceptibility of melanoma cells to TRAIL-induced killing (Hamai et al., 2006) and in our hands, treatment of infected macrophages with Imatinib for 120 hours removed the requirement for exogenous TRAIL for the induction of high level apoptosis in HIV-1 infected macrophages cultures. This suggests that Imatinib alone is sufficient to disrupt the balance of pro- and anti-apoptotic factors in infected macrophages. Mechanisms which restrict TRAIL-mediated apoptosis have been described for several unrelated viruses. Gamma-herpesviruses, including Kaposi's sarcomaassociated human herpesvirus-8, encode FLICE-inhibitory proteins (FLIPs) that interact with the adaptor protein, FADD which inhibits the generation of active caspase 8 that is necessary to trigger apoptosis by TRAIL (Thome et al., 1997). Human adenovirus type-5 encodes three proteins (RID) that induce the internalization from the cell surface and lysosomal degradation of TRAIL receptors (Tollefson et al., 2001). Human T cell leukemia virus type 1-infected T cell lines are resistant to TRAIL mediated apoptosis presumably due to activation of TRAIL expression by the viral transactivator Tax (Matsuda et al., 2005). We describe a novel mechanism for evasion of TRAIL dependent apoptosis, mediated by the HIV-1 envelope, by pathways that block TRAIL-R1 expression and inhibit downstream apoptotic signaling. Firstly, envelope induces M-CSF production which in turn, maintains TRAIL-R1 expression at close to uninfected cell levels, and secondly, envelope induces over-expression of anti-apoptotic genes, Mcl-1, Bfl-1, cIAP-1, cIAP-2 and XIAP, that function in preventing mitochondrial and caspase-mediated apoptosis by TRAIL (Salvesen and Duckett, 2002; Zhang & Fang, 2005; Han et al., 2006).

The work presented herein indicates that different strategies are used by HIV-1 to protect infected lymphocytes and infected macrophages from immune surveillance. In infected lymphocytes, HIV-1 inhibits the Fas and TNFα signaling pathways while simultaneously enhancing Fas expression to induce bystander killing of cytotoxic lymphocytes (Geleziunas et al., 2001; Fackler & Baur, 2002). Therefore, the immune evasion strategy in infected lymphocytes involves not only protecting infected cells from death signals but also in using these death signals to counter-attack HIV-1 specific cytotoxic T lymphocytes (Mueller et al., 2001). However, in the setting of viral dissemination in trans, this strategy would be counterproductive since efficient virus dissemination from infected macrophages to neighboring lymphocytes requires that the viability of both the donor and recipient cell must be preserved. The results presented herein offer one explanation for how HIV-1 has solved this problem, i.e. by conferring resistance to apoptotic signals provided by lymphocyte contact rather than up regulating death signals that kill neighboring lymphocytes (we did not observe any induction of TRAIL, TWEAK or Fas ligand expression on HIV-1-infected macrophages). Therefore, HIV-1 envelope-mediated regulation of cytokine, anti-apoptotic genes and death receptor expression may play dual roles in viral replication firstly, by allowing infected macrophages to withstand negative signals engendered by lymphocytes during infection in trans and, secondly, by allowing infected macrophages to evade the host apoptotic response.

### Methods

### Antibodies and Reagents

Neutralizing antibody to human M-CSF and Goat polyclonal antibodies were used at 10 μg/ml and obtained from R & D Systems, (Minneapolis, Minn.). ELISA kits for human MCSF were obtained from R & D Systems. HSA (murine CD24), TRAIL, Fas and TWEAK-R antibodies for flow cytometry and antibodies to CD3 and CD28 were obtained from BD Pharmingen (San Diego Calif.). TRAIL DR4 antibodies and soluble human recombinant TRAIL were obtained from Axxora LLC, (San Diego Calif.). Recombinant soluble Fas, TRAIL, TWEAK and control receptor proteins were obtained from R & D Systems. Monoclonal and polyclonal antibodies to the M-CSF receptor and antibody to phosphotyrosine were obtained from Santa Cruz Biotech (Santa Cruz Calif.). Imatinib mesylate was obtained from Sequoia Research Products (Pangbourne UK).

### Cells and Viruses

The X4 & R5 tropic viruses HIV-1 LAI, HIV-1 ADA and HIV-1 HSA were prepared as detailed (Swingler at al., 2003). HIV-1 HSA (Jamieson & Zack, 1998) contains a mouse gene CD24, heat stable surface antigen in place of Vpr. As a result, HIV-1 HSA infected cells can be identified by flow cytometry and pseudotyping with VSV-G envelope effectively by-passes the requirement for Vpr in macrophage infection. Envelope minus HIV-1 LAI and HIV-1 HSA variants were constructed by Nde I restriction site fill in. HIV-1 LAI ΔCD4b contains a two amino acid deletion in a critical CD4 receptor binding domain of HIV-1 envelope that disrupts CD4 binding (Thali et al, 1991). To obtain VSV envelope pseudotyped HIV-1 stocks, 25 μg HIV-1 DNA was co-transfected with 25 μg of a VSV-G expression plasmid. Lymphocytes and monocytes were obtained by leukapheresis from normal donors seronegative for HIV-1 and Hepatitis B. Monocytes were further separated by counter-current centrifugal elutriation as detailed elsewhere (Gendelman et al., 1988). Populations of T lymphocytes or CD8+ T cells were obtained by additional purification with antibody coated magnetic beads according to manufacturer's instructions (Dynal-Invitrogen, Carlsbad Calif.). Elutriated monocytes were cultured for 4 days in medium containing M-CSF (R & D Systems) and for a further 3 days in medium lacking M-CSF, and then used for virus infections. T lymphocytes were activated with antibodies to CD3 and CD28 (5 μg·ml-1 each) for 48 hours then washed in medium prior to co-culture with macrophages.

### Apoptosis, Cell Death and Viability Assays

ELISA for Caspase-3 cleavage (to active form) was obtained from Cell Signaling Technologies (Beverley Mass.). Briefly, 1.4×10⁶ macrophages infected with WT and envelope-minus viruses were lysed according to the supplier's protocol, 1 hour after the addition of soluble recombinant Trail (100 ng ml-1). Protein content was determined by Bradford Assay (Bio-Rad, Hercules Calif.) and 220 μg of protein in 200 μl from each culture was assayed. ELISA for supernatant release of histone associated DNA fragments released after induced cell death (Roche, Nutley N.J.) was used to measure macrophage longevity. Briefly, after the addition of soluble TRAIL from 0.7×10⁶ cells 10 μl of supernatant was harvested over time for ELISA. Supernatant from macrophages treated for 72 hrs with 100 μM Apoptosis Activator I (EMD biosciences, San Diego Calif.) which resulted in maximum cell death was used as a positive control and the value for viable cells calculated by subtraction. In some experiments an MTT assay (Sigma, St. Louis Mo.) was used to measure cell viability. Fluorescent detection of active caspases was performed by pre-treating cells with red-VAD-FMK and flow cytometry according to the manufacturer's protocol (EMD biosciences). For the measurement of apoptosis by Annexin V and propidium iodide staining in flow cytometry reagents were obtained from BioVision (Mountain View Calif.) and used according to the supplier's protocol.

### RNA Interference and Quantitation

Macrophages, 1.4×10⁶, were infected a wild-type HIV-1 ADA variant and monitored by RT assay until viral replication approached peak levels. Cells were transfected with Lipofectamine 2000 containing 100 nM each duplexed siRNA to cIAP-1, cIAP-2, XIAP, Bfl-1, Mcl-1 or scrambled siRNA obtained from Dharmacon (Lafayette, Colo.) for 4 hours then re-fed conditioned medium from uninfected macrophages. The transfection was repeated the next day. Sixteen hours following the second transfection, cells were harvested for ELISA, FACS or RNA analysis. FACS analysis was performed as described previously (Swingler et al., 2003). RNAi-mediated mRNA decay was assessed on total RNA from 200,000 cells prepared using Trizol (Invitrogen, Carlsbad Calif.) by SyBr Green real time RT-PCR (Quantitect Sybr Green kit; Qiagen, Valencia Calif.) using gene-specific primers from Superarray (Frederick Md.). The levels of cellular mRNAs were similarly determined by SyBr Green real time PCR or by cDNA gene arrays (Superarray) as described previously (Swingler et al., 2003).

Infection in Trans, T Cell Mediated Inhibition of Viral Replication and Induction of Macrophage Apoptosis

Infected macrophages were monitored by RT assay until viral replication approached peak. M-CSF was neutralized in infected cultures by the addition of Anti-M-CSF polyclonal antibody or isotype control for 24 hours. RNA interference was employed to silence cIAP-1, cIAP-2, XIAP, Bfl-1 and Mcl-1 genes and timed such that antibody was added after the second siRNA transfection as required. The medium was changed and supplemented with fresh antibody and 5 million Anti-CD3/CD28 stimulated autologous T lymphocytes. In some experiments, T lymphocytes were incubated with recombinant soluble TRAIL receptor proteins for 1 hour prior to co-culture with infected macrophages. After a 4 hour co-culture, lymphocytes were gently removed from the wells. Cellular DNA was extracted by DNAzol (Invitrogen, Carlsbad Calif.) and viral 2-LTR circles and CCR5 copy numbers determined by quantitative PCR (Sharkey et al., 2000). For the inhibition of HIV-1 replication, anti-CD3/CD28 stimulated autologous CD8+ T cells (2.5 million) were co-cultured with infected macrophages for 4 hours, gently removed and virus production determined by RT 24 hours later. The activation of macrophage caspases were measured after 4 hours co-culture with 5 million anti-CD3/CD28 stimulated autologous T cells by flow cytometry using a fluorescent peptide, red-VAD-FMK, as described above.

### REFERENCES

The following references, to the extent that they provide exemplary procedural or other details supplementary to those set forth herein, are specifically incorporated herein by reference.
Barber, G. N. Host defense, viruses and apoptosis. Cell Death Differ. 8, 116-26 (2001).
Bilim, V. et al. Role of XIAP in the malignant phenotype of transitional cell cancer (TCC) and therapeutic activity of XIAP antisense oligonucleotides against multidrug-resistant TCC in vitro. Int. J. Cancer 103 29-37 (2003).
Butte, A., Ye, J., Haring, H., Stumvoll, M., White, M., et al., Determining significant fold differences in gene expression analysis. Pac Symp Biocomput: 6-17 (2001).
Carr, J. M., Hocking, H., Li, P. & Burrell, C. J. Rapid and efficient cell-to cell transmission of human immunodeficiency virus infection from monocyte-derived macrophages to peripheral blood lymphocytes. Virology 265, 319-29 (1999).
Chan, S. L. et al. Identification of chelerythrine as an inhibitor of BclXL function. J. Biol. Chem. 278 20453-20456 (2003).
Cicala, C. et al. Induction of phosphorylation and intracellular association of CC chemokine receptor 5 and focal adhesion kinase in primary human CD4+ T cells by macrophage-tropic HIV envelope. J. Immunol. 163, 420-6 (1999).
Cicala, C. et al. HIV envelope induces a cascade of cell signals in non-proliferating target cells that favor virus replication. Proc. Natl. Acad. Sci. USA 99, 9380-5 (2002).
Davis, C. B. et al. Signal transduction due to HIV-1 envelope interactions with chemokine receptors CXCR4 or CCRS. J. Exp. Med. 186, 1793-8 (1997).
Degterev, A. et al. Identification of small-molecule inhibitors of interaction between the BH3 domain and Bcl-xL. Nat. Cell Biol. 3 173-182 (2001).
Derouet, M., Thomas, L., Cross, A., Moots, R. J. & Edwards, S. W. Granulocyte macrophage colony-stimulating factor signaling and proteasome inhibition delay neutrophil apoptosis by increasing the stability of Mcl-1. J. Biol. Chem. 279, 26915-21 (2004).
Enyedy, I. J. et al. Discovery of small-molecule inhibitors of Bcl-2 through structure-based computer screening. J. med. Chem. 44 4313-4324 (2001).
Fackler, 0. T. & Baur, A. S. Live and let die: Nef functions beyond HIV replication. Immunity 16; p493-7 (2002).
Fulda, S. et al. Smac agonists sensitize for Apo2L/TRAIL- or anticancer drug-induced apoptosis and induce regression of malignant giloma in vivo. Nat. Med. 8 808-815 (2002).
Geleziunas, R., Xu, W., Takeda, K., Ichijo, H. & Greene, W. C. HIV-1 Nef inhibits ASK 1-dependent death signaling providing a potential mechanism for protecting the infected host cell. Nature 410, 834-8 (2001).
Gendelman, H. E. et al. Efficient isolation and propagation of human immunodeficiency virus on recombinant colony-stimulating factor 1-treated monocytes. J. Exp. Med. 167, 1428-41 (1988).
Gooding, L. R., Elmore, L. W., Tollefson, A. E., Brady, H. A. & Wold, W. S. A 14.700 MW protein from the E3 region of adenovirus inhibits cytolysis by tumor necrosis factor. Cell 53, 341-6 (1988).
Gruber, M. F., Weih, K. A., Boone, E. J., Smith, P. D. & Clouse, K. A. Endogenous macrophages CSF production is associated with viral replication in HIV-1 infected human monocyte-derived macrophages. J. Immunol. 154, 5528-36 (1995).
Hamai, A., et al. Imatinib enhances human melanoma cell susceptibility to TRAIL-induced cell death: relationship to Bcl-2 family and caspase activation. Oncogene 25, 7618-34 (2006).
Han, J., Goldstein, L., Gastman. Rabinowich, H. Interrelated roles for Mcl-1 and BIM in regulation of TRAIL-mediated mitochondrial apoptosis. J. Biol. Chem. 281, 10153-63 (2006).
Holinger, E. P. et al. Bak BH3 peptides antagonize Bcl-xL function and induce apoptosis through cytochrome c-independent activation of caspases. J. Biol. Chem. 274 13298-13304 (1999).
Hu, Y. et al. Antisense oligonucleotides targeting XIAP induce apoptosis and enhance chemotherapeutic activity against human lung cancer cells in vitro and in vivo. Clin. Cancer Res. 9 2826-2836 (2003)
Igarashi, T. et al. Macrophages are the principal reservoir and sustain high virus loads in rhesus macaques after depletion of CD4+ T cells by a highly pathogenic simian immunodeficiency virus/HIV type 1 chimera (SHIV): implications for HIV-1 infection of humans. Proc. Natl. Acad. Sci. USA 98, 658-63 (2001).
Jamieson, B. D. & Zack, J. A. In vivo pathogenesis of a human immunodeficiency type 1 reporter virus. J. Virol. 72, 6520-6 (2003).
Kaplan, M. J. et al. TRAIL (Apo2 Ligand) and TWEAK (Apo3 Ligand) mediate CD4+ T cell killing of antigen-presenting macrophages. J. Immunol. 164, 2897-904 (2000).
Kayagaki, N. et al. Involvement of TNF-related apoptosis-inducing ligand in human CD4+ T cell-mediated cytotoxicity. J. Immunol. 162, 2639-47 (1999).
Koka, P et al. The mapping of HIV-1 gp160 epitopes required for interleukin-1 and tumor necrosis factor α production in glial cells. J Neuroimmunol. 57; p179-91 (1995).
Kuroda, L. et al. Bim and Bad mediate Imatinib-induced killing of Bcr/Abl+ leukemic cells, and resistance due to their loss is overcome by a BH3 mimetic. Proc. Natl. Acad. Sci. USA 40, 14907-12 (2006).
Kutza, J. et al. Macrophage colony-stimulating factor antagonists inhibit replication of HIV-1 in human macrophages. J. Immunol. 164 4955-60 (2000).
Li, L. et al. A small molecule Smac mimic potentiates TRAIL- and TNFalpha-mediated cell death. Science 305 1471-1474 (2004).
Li, W. Q., Jiang, Q., Khaled, A. R., Keller, J. R. & Durum, S. K. Interleukin-7 inactivates the pro-apoptotic protein Bad promoting T cell survival. J. Biol. Chem. 279, 29160-6 (2004).
Lum, J. J. et al. Induction of cell death in human immunodeficiency virus-infected macrophages and resting memory CD4 T cells by TRAIL/Apo21. J. Virol. 75, 11128-36 (2001).
Martoglio, B., Graf R. & Dobberstein, B. Signal peptide fragments of preprolactin and HIV-1 p-gp160 interact with calmodulin. EMBO J. 16; p6636-45 (1997).
Matsuda, T., et al. Resistance to Apo2 ligand (Apo2L)/tumor necrosis factor-related apoptosis-inducing ligand (TRAIL)-mediated apoptosis and constitutive expression of Apo2L/TRAIL in human T-cell leukemia virus type 1-infected T-cell lines. J. Virol. 79, 1367-78 (2005).
McManus, D. C. et al. Loss of XIAP protein expression by RNAi and antisense approaches sensitizes cancer cells to functionally diverse chemotherapeutics. Oncogene 23 8105-8117 (2004)
Meuller, Y. M. et al. Increased CD95/Fas-induced apoptosis of HIV-specific CD8(+) T cells. Immunity 15, 871-82 (2001).
Micoli, K. J. et al. Requirement of calmodulin binding by HIV-1 gp160 for enhanced FAS-mediated apoptosis. J. Biol. Chem. 275; p 1233-40 (2000).
Mirandola, P. et al. Activated human NK and CD8+ T cells express both TNF-related apoptosis-inducing ligand (TRAIL) and TRAIL receptors but are resistant to TRAILmediated cytotoxicity. Blood 104, 2418-24 (2004).
Nikolovska-Coleska, Z. et al. Discovery of embelin as a cell-permeable, small-molecular weight inhibitor of XIAP through structure-based computational screening of a traditional herbal medicine three-dimensional structure database. J. Med. Chem. 47 2430-2440 (2004).
Oost, T. K. et al. Discovery of potent antagonists of the antiapoptotic protein XIAP for the treatment of cancer. J. Med. Chem. 47 4417-4426 (2004).
Perelson, A. S. et al. HIV-1 dynamics in vivo: virion clearance rate, infected cell lifespan and viral generation time. Science 271, 1582-6 (1996).
Raftery, M. J. et al. Targeting the function of mature dendritic cells by human cytomegalovirus: a multilayered viral defense strategy. Immunity 15, 997-1009 (2001).
Ray, N. & Doms, R. W. HIV-1 coreceptors and their inhibitors. Curr. Top. Microbiol. Immunol. 303, 97-120 (2006).
Richardson, B. C., Buckmaster, T., Keren, D. F. & Johnson, K. J. Evidence that macrophages are programmed to die after activating autologous antigen-specific CD4+ T cells. Eur. J. Immunol. 23, 1450-5 (1993).
Salvesen, G. S, and Duckett, C. S. IAP proteins: blocking the road to death's door. Nat. Rev. Mol. Cell. Biol. 3, 401-9 (2002).
Schimmer, A. D. et al. Small-molecule antagonists of apoptosis suppressor XIAP exhibit broad antitumor activity. Cancer Cell 5 25-35 (2004).
Secchiero, P. et al. Human herpesvirus 7 induces the functional up-regulation of tumor necrosis factor-related apoptosis-inducing ligand (TRAIL) coupled to TRAIL-R1 downrodulation in CD4+ T cells. Blood 98, 2474-81 (2001).
Sharkey, M. et al. Persistence of episomal HIV-1 infection intermediates in patients on highly active antiretroviral therapy. Nat. Medicine 6, 76-81 (2000).
Sharova, N., Swingler, C., Sharkey, M. & Stevenson M. Macrophages archive HIV-1 virions for viral dissemination in trans. EMBO J. 24, 2481-9 (2005).
Stevenson, M. HIV-1 pathogenesis. Nat. Medicine 9, 853-60 (2003).
Sun, H. et al. Structure-based design, synthesis, and evaluation of conformationally constrained mimetics of the second mitochondria-derived activator of caspase that target the X-linked inhibitor of apoptosis protein/caspase-9 interaction site. J. Med. Chem. 47 4147-4150 (2004).
Swingler, S. et al. HIV-1 Nef mediates lymphocyte chemotaxis and activation by infected macrophages. Nat. Medicine 5, 997-1003 (1999).
Swingler, S. et al. HIV-1 Nef intersects the macrophage CD40L signaling pathway to promote resting-cell infection. Nature 424, 213-9 (2003).
Taylor, J. R., Brownlow, N., Domin, J. & Dibb, N. J. FMS receptor for M-CSF (CSF-1) is sensitive to the kinase inhibitor imatinib and mutation of Asp-802 to Val confers resistance. Oncogene 25, 147-51 (2006).
Thali, M. et al. Effects of changes in gp 120-CD4 binding affinity on Human Immunodeficiency Virus Type 1 envelope glycoprotein function and soluble CD4 sensitivity. J. Virol. 65; p5007-12 (1991).
Thome, M., et al. Viral FLICE-inhibitory proteins (FLIPs) prevent apoptosis induced by death receptors. Nature 386, 517-21 (1997).
Tollefson, A. E. et al. Forced degradation of Fas inhibits apoptosis in adenovirus infected cells. Nature 392, 726-30 (1998).
Tollefson, A. E. et al. Inhibition of TRAIL-induced apoptosis and forced internalization of TRAIL receptor by Adenovirus proteins. J. Virol. 75, 8875-87 (2001).
Tzung, S. P. et al. Antimycin A mimics a cell-death-inducing Bcl-2 homology domain 3. Nat. Cell Biol. 3 183-191 (2001).
Walensky, L. D. et al. Activation of apoptosis in vivo by a hydrocarbon-stapled BH3 helix. Science 305 1466-1470 (2004).
Wang, J. L. and Liu D. et al. Structure-based discovery of an organic compound that binds Bcl-2 protein and induces apoptosis of tumor cells. Proc. Natl. Acad. Sci. USA. 97 7124-7129 (2000^{A}).
Wang, J. L. and Zhang Z. J. et al. Cell permeable Bcl-2 binding peptides: a chemical approach to apoptosis induction in tumor cells. Cancer Res. 60 1498-1502 (2000^{B}).
Weissman, D. et al. Macrophage-tropic HIV and SFV envelope proteins induce a signal through the CCR5 chemokine receptor. Nature 389, 981-5 (1997).
Wu, T. Y. et al. Development and characterization of nonpeptidic small molecule inhibitors of the XIAP/caspase-3 interaction. Chem. Biol. 10, 759-767 (2003)
Yang, L. et al. Predominant suppression of apoptosome by inhibitor of apoptosis protein in non-small cell lung cancer H460 cells: therapeutic effect of a novel polyarginine-conjugated Smac peptide. Cancer Res. 63 831-837 (2003).
Zangemeister-Wittke, U. et al. A novel bispecific antisense oligonucleotide inhibiting both bcl-2 and bcl-xL expression efficiently induces apoptosis in tumor cells. Clin. Cancer Res. 6 2547-2555 (2000).
Zhang, L. & Fang, B. Mechanisms of resistance to TRAIL-induced apoptosis in cancer. Cancer Gene Therapy 12, 228-37 (2005).

### EQUIVALENTS

Those skilled in the art will recognize, or be able to ascertain using no more than routine experimentation, many equivalents of the specific embodiments of the invention described herein. Such equivalents are intended to be encompassed by the following claims.

We claim:

1. A method for inducing apoptosis in an HIV reservoir cell, the method comprising contacting the HIV reservoir cell with an effective amount of an M-CSF effector kinase inhibitor for inhibiting M-CSF signaling.

2. The method of claim 1, wherein the reservoir cell is a macrophage.

3. The method of claim 1, further comprising contacting the HIV reservoir cell with a TRAIL molecule.

4. The method of claim 3, wherein the TRAIL molecule is a TRAIL agonist.

5. The method of claim 1, wherein the M-CSF effector kinase inhibitor does not directly inhibit HIV replication.

6. The method of claim 1, wherein the M-CSF effector kinase inhibitor is selected from the group consisting of:
Imatinib mesylate; ST1571; Nilotinib; Dasatinib; Sorafenib; Sunitinib; GW2580, 5-{3-methoxy-4-[(4methoxybenzyl)oxy]benzyl}pyrimidine-2,4-diamine; ABT-869; AG013736; BAY 43-9006; CHIR258; SU11248.

7. The method of claim 4, further comprising contacting the reservoir cell with a histone deacetylase inhibitor.

8. The method of claim 7, wherein the histone deacetylase inhibitor is selected from the group consisting of:
valproic acid (VPA), sulforaphane, suberoylanilide hydroxamic acid (SAHA), sodium n-butyrate, suberoylanilide hydroxamic acid, LAQ824, CI-994, MS-275, and depsipeptide.

9. A method for treating a subject having an HIV infection, the method comprising administering to a subject in need of such treatment an effective amount of an M-CSF effector kinase inhibitor for inhibiting M-CSF signaling, wherein the effective amount of M-CSF effector kinase inhibitor is not an effective amount for inhibiting HIV replication.

10. The method of claim 9, wherein the effective amount of an M-CSF effector kinase inhibitor induces TRAIL-mediated apoptosis in an HIV reservoir cell and causes a reduction in a number of HIV reservoir cells in the subject.

11. A method for treating a subject having an HIV infection, the method comprising administering to a subject in need of such treatment an effective amount for treating HIV of a histone deacetylase inhibitor and a TRAIL receptor activator.

12. The method of claim 11, wherein the TRAIL receptor activator is a M-CSF antagonist.

13. The method of claim 12, wherein the M-CSF antagonist is an antibody to M-CSF.

14. The method of claim 12, wherein the M-CSF antagonist is a siRNA for M-CSF.

15. The method of claim 12, wherein the M-CSF antagonist is an M-CSF effector kinase inhibitor.

16. The method of claim 11, wherein the TRAIL receptor activator is a TRAIL receptor agonist.

17. The method of claim 11, wherein the TRAIL receptor activator is a TRAIL receptor expression construct.

18. The method of claim 11, wherein the histone deacetylase inhibitor is selected from the group consisting of:
valproic acid (VPA), sulforaphane, suberoylanilide hydroxamic acid (SAHA), sodium n-butyrate, suberoylanilide hydroxamic acid, LAQ824, CI-994, MS-275, and depsipeptide.

19. A method of upregulating cell surface death receptors in an HIV-infected reservoir cell, the method comprising:
contacting the HIV-infected reservoir cell with an effective amount of an M-CSF antagonist to upregulate cell surface death receptors, and
contacting the cell with a TRAIL ligand or an agonist thereof.

20. The method of claim 19, wherein the M-CSF antagonist is selected from the group consisting of:
an M-CSF antibody, siRNA for M-CSF, Imatinib mesylate; ST1571; Nilotinib; Dasatinib; Sorafenib; Sunitinib; GW2580, 5-{3-methoxy-4-[(4methoxybenzyl)oxy]benzyl}pyrimidine-2,4-diamine; ABT-869; AG013736; BAY 43-9006; CHIR258; SU11248.

21. The method of claim 19, wherein the TRAIL ligand or agonist thereof is a TRAIL receptor antibody.

22. A method for treating a subject having an HIV infection by selectively inducing TRAIL-mediated apoptosis in an HIV-infected cell, the method comprising administering to a subject in need of such treatment a TRAIL receptor activator.

23. The method of claim 22, wherein the TRAIL receptor activator is a TRAIL receptor gene expression construct.

24. The method of claim 22, wherein the TRAIL receptor activator is a topoisomerase II inhibitor.

25. The method of claim 24, wherein the topoisomerase II inhibitor is Etoposide or teniposide.

26. The method of claim 9, further comprising administering to the subject one or more agents that regulate one or more targets selected from the group consisting of:
Death receptors, CD95/Fas, TNF, Caspases, IAPs/SMAC, Bcl-2 and p53.


SEQUENCE LISTING (1 sequence)

SEQ ID NO: 1 - protein, 5 aa, Artificial sequence
  Synthetic polypeptide
GGGGS